(19) United States
(12) Patent Application Publication
(10) Pub. No.: US 2021/0236845 A1
(43) Pub. Date: Aug. 5, 2021

(54) RETINAL STIMULATOR

(71) Applicant: The Regents of the University of California, Oakland, CA (US)

(72) Inventors: Yi-Ren Ng, Berkeley, CA (US); Austin Roorda, Berkeley, CA (US); Brian Schmidt, Berkeley, CA (US); Utkarsh Singhal, Berkeley, CA (US)

(21) Appl. No.: 17/235,627

(22) Filed: Apr. 20, 2021

Related U.S. Application Data
(63) Continuation of application No. PCT/US2019/057478, filed on Oct. 22, 2019.
(60) Provisional application No. 62/748,944, filed on Oct. 22, 2018.

(51) Int. Cl. A61N 5/06; G06T 7/00

(52) U.S. Cl. CPC A61N 5/0622; A61N 5/062; A61N 2005/067; A61N 2005/0661; A61N 2005/0663; G06T 7/0012

(57) ABSTRACT

According to various embodiments of the invention, systems and methods for stimulating the retina are presented, by a combination of some or all of the following: imaging and mapping the retina; determining or assigning parameters on the retinal map, including real and virtual cell types and properties; tracking the head and/or eye; receiving or creating, transforming and/or projecting desired image signal onto the retinal map; combining the desired image with retinal parameter map data in order to determine per-cell or per-retinal-location stimulus values; and delivering the desired stimulus values to the retina. According to various embodiments of the invention, improved or novel color display, vision and perception are achieved: color gamut that encompasses the full human gamut; gamut that goes beyond the full human gamut; providing trichromatic vision functionality and/or color perception to dichromats, monochromats and/or individuals with anomalous color vision; providing perception of N-dimensional color, where N is higher than 3.

### FEDERALLY SPONSORED RESEARCH AND DEVELOPMENT

This invention was made with government support under Grant EY023591 awarded by the National Institute of Health and Grant 1617794 awarded by the National Science Foundation. The government has certain rights in the invention.

### FIELD OF THE INVENTION

Various embodiments of the present invention relate to: display of images, imaging; motion tracking; retinal imaging, tracking, stabilization and stimulation; cell identification and characterization; color gamut, appearance and perception. More particularly, certain embodiments of the present invention relate to presenting imagery to a viewer by stimulating the retinal cells in various ways to extend the space, dimension, resolution and/or quality of colors that may be perceived.

### BACKGROUND

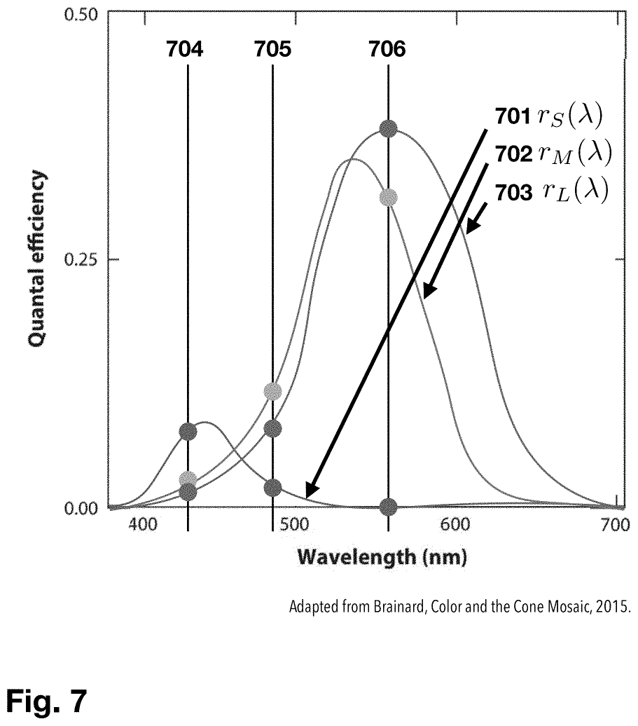

Mammal-like eyes have a retina that contains various types of photosensitive cells. For example, in the human retina these cells include so-called S, M and L cone cells (the cells most closely associated with color vision, and which have photoresponse functions that are concentrated in the, respectively, short, medium and long wavelengths). Referring to FIG. 7, these S, M and L cone cells have spectral response functions, respectively, r_{S}(λ), r_{M}(λ) and r_{L}(λ) of wavelength λ, centered in the short, medium and long wavelengths, and depicted by graphs 701, 702 and 703 in the figure. These functions define the likelihood that the cone cells fire in a neuronal sense as a result of incident light of a given power, as a function of wavelength. It is well known that there is no spectral power distribution that will stimulate only the M cone cells, because r_{M}(λ) overlaps with r_{S}(λ) and/or r_{L}(λ) for all λ for which r_{M}(λ) is non-zero. For example, see the wavelengths marked at positions 704, 705 and 706 on the figure. More generally, the responses s, m and l of neighboring S, M and L cone cells close to a position on the retina is naturally constrained to satisfy

s=P(λ)r_{S}(λ)dλ (1)

m=P(λ)r_{M}(λ)dλ (2)

l=P(λ)r_{L}(λ)dλ, (3)

where P(λ) is a real spectral power distribution—a non-negative function that represents the amount of light incident on the cell of interest for each wavelength λ.

One of the ideas underlying various embodiments of the present invention is to use single-cell targeting capability to deliver precise stimulation values s*, m* and l* to the S, M and L cells—values that are freed from the natural constraints described in Equations 1-3 above. Delivering values that violate those natural constraints may give rise to an extended gamut of neurally perceived colors. For example, one stimulus value that falls outside of the natural human color gamut is stimulating the M cells exclusively, for example, generating an (s, m, l) retinal response of (0, 1, 0) in effective relative intensity, which cannot occur in normal vision. In contrast, the analogous triplets of (1,0,0) and (0,0,1) relative intensity may occur in natural viewing, for example as a response to viewing monochromatic light of the shortest and longest visible wavelengths, respectively, and give percepts of red and blue colors of high saturation.

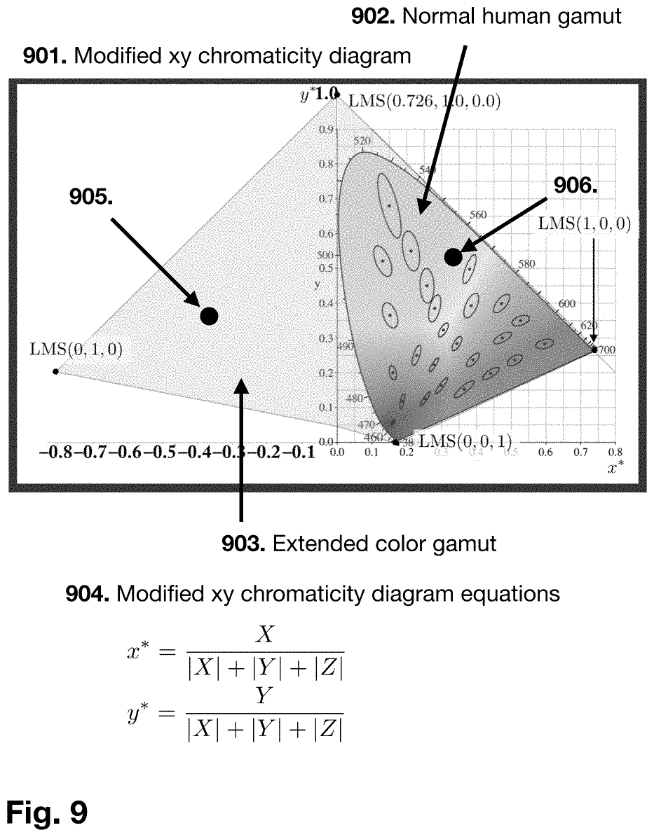

A well-known way to visualize the limitations on the perceived gamut is depicted in FIG. 9, which shows an x*y* chromaticity diagram that is closely related to the common CIE 1931 xy chromaticity (see Fairchild, 2013). The equations 904 for the coordinates of x*y* differ from conventional xy in the use of absolute values for the CIE XYZ coordinate values in the denominator of the equations. However, since X,Y,Z are positive within the full human gamut the two chromaticity diagrams are identical within the well-known horseshoe-shaped gamut (902) of normal human color vision. Note that there exists no possible lights in natural viewing that can produce chromaticities outside the bounds of this horseshoe-shaped gamut. In contrast, various embodiments of the present invention break this fundamental limitation and enable stimulation of chromaticities defined by the enlarged color gamut enclosed by the polygon 903 that is a superset of the horseshoe-shaped natural gamut.

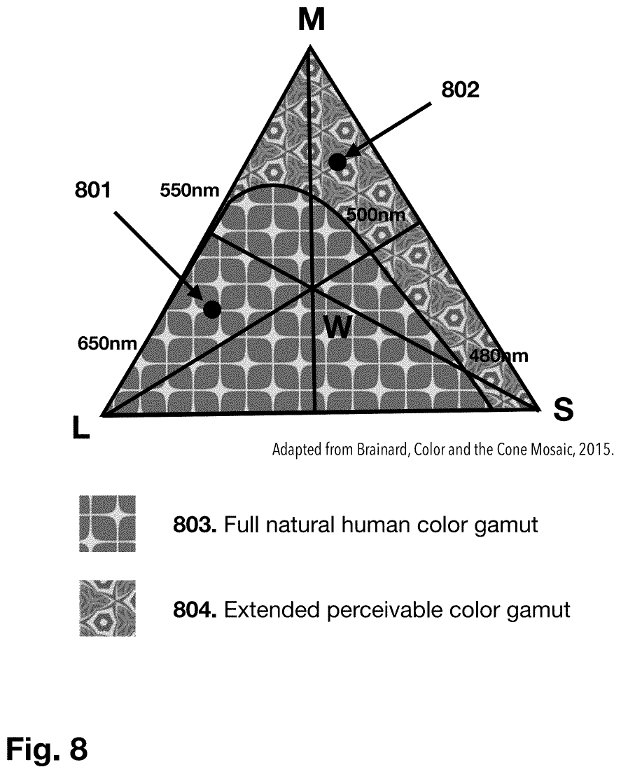

FIG. 8 depicts another way to visualize the limited natural gamut 803, and enlarged color gamut 804. This triangular chromaticity diagram depicts stimuli with relative ratios (S,M,L) of the three cone cells on the retina as a point at the corresponding barycentric coordinates within the triangle.

### SUMMARY

According to various embodiments of the present invention, the aforementioned and other limitations are lifted as relating to displaying and causing a viewer to perceive imagery of various sorts. The present invention is exemplified in a number of illustrative embodiments, implementations and applications, which are summarized below.

In various exemplary embodiments of the present invention, the goal is to compute and deliver specific light micro-doses to photosensitive cells in the retina, in order to create a desired visual percept. In certain embodiments, this enables creation of full color percepts that span the full human color gamut, rather than the restricted color gamut of conventional displays that superimpose red, green and blue (RGB) pixels. In other embodiments of the present invention, a visual percept is created that contains colors beyond the natural human color gamut, to include colors that cannot be perceived when viewing the natural world. In yet other exemplary embodiments, the light micro-doses to photosensitive cells are designed so as to enable a color-blind person (e.g. a deuteranope) to achieve full trichromatic color vision function. In yet other embodiments, the retinal signal enables the user, possibly after an acclimation and learning phase, to functionally achieve N-dimensional color vision, where N is greater than 3. In yet other embodiments of the present invention, colors can be displayed to the user that are perceptually more saturated (or have higher colorfulness) than colors in the natural human gamut; in one of these exemplary embodiments, the system stretches the saturation (or colorfulness) of input imagery so that it extends into the higher, non-natural saturation (or colorfulness) levels, which may therefore be perceived by the user as higher saturation or colorfulness than regular imagery.

In various exemplary embodiments of the present invention, the desired light micro-doses for photosensitive cells may be computed as the output of a processing system. Various functions and characteristics of this processing system are now discussed in the context of an exemplary embodiment to elucidate the ideas. One module of the system images and maps the retina and geometry (e.g. location, shape and/or size) of the photosensitive cells. Upon this map, and according to the specific desired display application, certain parameters are assigned to various retinal positions and at cells located on the retinal map. In an exemplary embodiment, a module receives or creates a desired image signal, such as a color video signal, to be displayed to the user. Another module tracks the head and/or eye movements of the user, which may vary as a function of time, such that the position and gazing direction of the eye relative to the world may be determined. This information about the tracked head and eye geometry is used, in concert with the desired image signal, to compute the corresponding desired stimulus values for photosensitive cells in the retina. For example, color video signal may be converted to the color space defined by the spectral response functions of the S, M and L cone cells.

In various exemplary embodiments of the present invention, the previously computed, desired micro-doses for photosensitive cells, are physically delivered to the cells in the retina. For example, in certain embodiments, this module 207 may be an optical system comprising an imaging and tracking system of the retina to locate the position of cells in realtime, and a scanned laser raster beam that is modulated as a function of position in order to deliver the desired micro-dose as it scans over a cell. In a specific exemplary embodiment this optical system may be, or may include, an adaptive optics scanning laser ophthalmoscope (further implementation details of these exemplary embodiments are described below in sections related to AOSLO, eye-tracking, ITRACK and RetCon.

The preceding summary is not intended to describe every embodiment, implementation or application of the present inventions. The drawings and detailed description below further exemplify various embodiments of the present invention.

### DETAILED DESCRIPTION

Various embodiments of the present invention include some or all of the following steps, or subsystems that perform the following steps:

Mapping the retina, potentially including the position and/or size and/or shape of various photosensitive cells.

Assigning real or virtual parameters over the retinal map.

Tracking head and/or eye movement.

Receiving or creating desired image signal to be displayed.

Transforming and/or projecting the image signal to the retinal surface.

Calculating stimulus values for individual photosensitive cells as a function of the retinal parameter maps and desired image signal.

Physically delivering the desired per-cell stimulus values to the cells.

It should be understood that these steps may be implemented in various ways, alternatives and variants, in order to suit the specific desired application, and all such ways, alternatives and variants are intended as part of the present invention. Specific example embodiments of these ways, alternatives and variants are described in detail below in order to explain the main concepts.

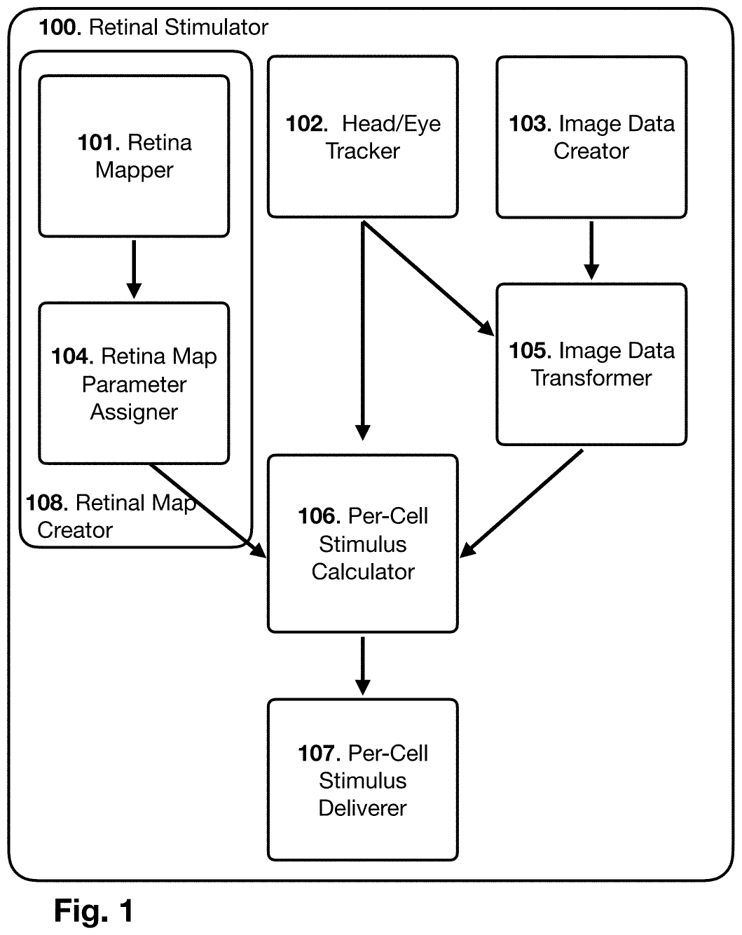

Referring to FIG. 1, the drawing shows an architecture for implementing the present invention according to one exemplary embodiment in the context of a Retinal Stimulator 100, which is summarized here. A Retina Map Creator 108 is comprised of a Retina Mapper 101 and Retina Map Parameter Assigner 104, which, in certain embodiments, creates a map of real, virtual, computed and/or derived parameters over the retinal surface. The Retina Mapper 101 maps the retina and aspects of the geometry (e.g. location, shape and/or size) of the photosensitive cells. On the resulting retinal map, a Retina Map Parameter Assigner 104 then assigns certain parameters to various positions and cells within the map. For example, this could include the biological (S, M, L) type of each cone cell. A Head/Eye Tracker 102 tracks the position of the eye and retina relative to the desired image to be displayed, usually continuously varying as a function of time. An Image Data Creator 103 is the source of imagery that is desired to be displayed to the user, also usually continuously varying as a function of time. An Image Data Transformer 105 receives the desired imagery and the head/eye tracking information, and computes a transformation and/or projection of the desired imagery onto the retinal map. A Per-Cell Stimulus Calculator 106 receives the retinal parameter map and the imagery transformed onto the retinal map, and calculates desired stimulus values for various photosensitive cells on the retina. A Per-Cell Stimulus Deliverer 107 receives the desired values for various cells and physically delivers the stimulus to the cells in the retina.

In various embodiments of the present invention, the Retina Mapper 101 may be implemented in any number of ways currently known or invented in the future. Certain exemplary embodiments may use an adaptive optics scanning laser ophthalmoscope to image the retina at a level that permits individual retinal cells to be discerned, and exemplary implementations of these kinds of embodiments are described in detail below.

In various embodiments of the present invention, the Retina Map Parameter Assigner 104 may assign to photosensitive cells in the retinal map various parameters including but not limited to the following: the biological type (e.g. S, M, L) of various photosensitive cells; virtual photoreceptor types: and virtual spectral responsivity curves.

In various embodiments of the present invention, the Head/Eye Tracker 102 may be implemented in whole or in part by methods that include, but are not limited to: using an off-the-shelf six degree-of-freedom head tracker, such as used in virtual-reality headsets; using an off-the-shelf eye-tracking system, such as those shining an infrared (IR) light source on the cornea and imaging the reflection to infer eye-gaze geometry; a retinal imaging system and position-inference system that infers the movement of the retina by tracking the displacement of the pattern of retinal cells; an adaptive optics scanning laser ophthalmoscope that images the retina at cellular resolution and tracks its motions, including drift, micro-saccades, tremors and saccades, as described in further detail below.

The Image Data Creator 103 may generate desired imagery of a multitude of subjects and a multitude of formats, according to myriad applications. Examples include, but are not limited to: RGB color video of the world; hyper-spectral video; renderings of 3D worlds using computer graphics in either RGB or hyper-spectral color; imagery that is grayscale, full color, or is comprised of a plurality of image channels.

The Image Data Transformer 105 may be implemented in various ways according to the geometry and representation of the supplied imagery and head/eye tracking data. In many embodiments of the present invention, the key function of the transformer is to project the imagery from world or display coordinates onto the coordinates of the retinal map. By way of example, and for the purpose of elucidating the ideas, consider an embodiment of the present invention in which (a) the imagery provided is equivalent to a spherical video around the user that is indexed by viewing direction in world coordinates, and (b) the head/eye tracker provides the location and gaze direction of the eye in world coordinates. In this exemplary embodiment, the transformer 105 may be implemented in a manner that is equivalent or that approximates, the following: consider each position of interest on the retina, ray-trace it out of the pupil into the world, and sample the spherical video imagery according to the resulting ray direction.

The Per-Cell Stimulus Calculator 106 has the purpose of computing the desired stimulus value for various cells in the retina. The desired output, and the associated computation, vary widely depending on the target application, and implementations for a wide variety of exemplary embodiments are depicted in the drawings and described in detail below. Here we summarize the inputs and output of a few of these exemplary embodiments. In one exemplary embodiment directed at reproducing a color percept of the desired image with full color gamut of human vision without restriction, the Calculator receives S,M,L channel imagery transformed to the retinal map, and a retinal parameter map that defines whether each cone cell is S, M or L; the Calculator outputs stimulus values for each cone cell that match the values those cells would have received if viewing the scene normally. In another exemplary embodiment, directed at color blind individuals, the Calculator receives S,M,L channel imagery transformed to the retinal map, and a retinal parameter map that defines whether each cone cell is S or L (assuming, without loss of generality, that the M-type of cone cell is missing); the Calculator outputs stimulus values for the S cone cells that match the S image channel, and distributes the M and L image channels over subsets of the L cone cells, thereby injecting full trichromatic color information into the retina and brain. In yet another exemplary embodiment directed at creating color perception of N-dimensions, where N is greater than 3, the Calculator receives N-channel imagery transformed to the retinal map, and a retinal parameter map that assigns each cone cell one of N virtual photoreceptor types corresponding to each of the N channels of the imagery; the Calculator outputs stimulus values for each cone cell that match the values in the image channel that corresponds to the virtual receptor type assigned to that cell.

The Per-Cell Stimulus Deliverer 107 has the purpose of delivering the desired stimulus values to the various photosensitive cells. This may be implemented in any number of ways currently known or invented in the future. Certain exemplary embodiments may use an adaptive optics scanning laser ophthalmoscope to image individual retinal cells, and deliver the desired dose to each cell by appropriately modulating the intensity of a visible spectrum laser as it repetitively scans over the retina and passes over each cell in question. Exemplary implementations of these kinds of embodiments are described in detail below.

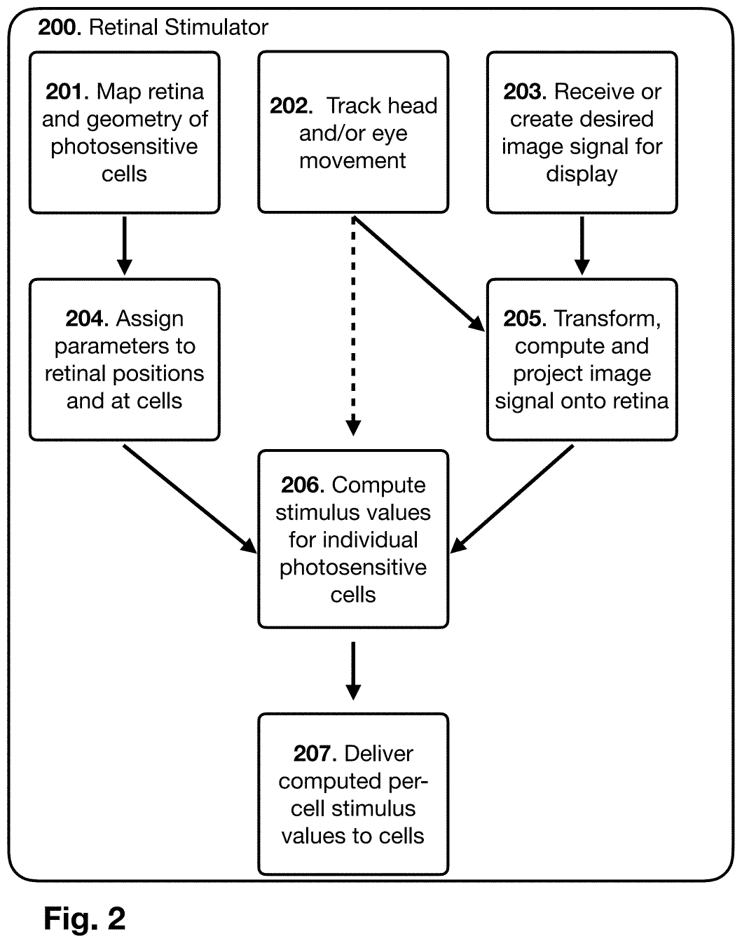

Referring now to FIG. 2, the drawing shows another architecture for implementing the present invention according to another exemplary embodiment. In this, as in various other embodiments, the desired light micro-doses for photosensitive cells for the Retinal Stimulator 200 may be computed as the output of a processing system. One module 201 of the architecture maps the retina and geometry (e.g. location, shape and/or size) of the photosensitive cells. While this may be accomplished in any number of ways currently known or invented in the future, certain exemplary embodiments may use an adaptive optics scanning and tracking laser ophthalmoscope to image the retina (further details are described elsewhere in this detailed description) at a level that permits individual retinal cells to be discerned. Another module 204 then assigns certain parameters to retinal positions and at cells imaged on the retina.

Another module 203 receives or creates a desired image signal to be displayed to the user. For clarity of understanding, one embodiment of this module creates an image signal that is a color video signal of the world surrounding the user. However, it should be understood that a wide range of image signal choices can be chosen according to the desired application; various other exemplary embodiments are described throughout this detailed description. Continuing the description of this exemplary embodiment, a module 202 tracks the head and/or eye movements of the user, which may vary as a function of time, such that the position and gazing direction of the eye relative to the world may be determined. While this may be accomplished in any number of ways currently known or invented in the future, in one exemplary embodiment this module works by having the user gaze at a fixed position in the world while an adaptive optics scanning laser ophthalmoscope is used to image and computationally track the movement of the retinal cone mosaic relative to the user's fixation point; the motion of the retinal mosaic relative to the fixation point is directly related to the eye movements of the user.

Continuing the description of the exemplary embodiment, another module 205 accepts the head and/or eye tracking geometry and the desired image signal, and calculates the transformation and/or projection of the image signal onto the retina represented by the retinal map. Further, module 206 computes the corresponding desired stimulus values for photosensitive cells in the retina. The computational function of this module 206 may be implemented in flexible and diverse ways according to desired application, and various exemplary embodiments are described throughout this detailed description. For clarity at this point, let us consider a specific exemplary embodiment, in which this module is implemented by considering the computed projection of the desired RGB color imagery onto the retinal surface, determining the location of the photosensitive cells relative to this projected imagery, and computing the value for each cell according to Equations 1-3 above (choosing the relevant equation according to the type of each cell), and where P(λ) is the spectral power distribution of the imagery incident on the cell in question. The result of this is to calculate the scalar stimulus value for cone cells that would reproduce the cone's stimulus in response to looking at the desired imagery in reality.

Continuing the description of the exemplary embodiment, a module 207 accepts the computed, desired micro-doses for photosensitive cells, and physically delivers them to the cells in the retina. This may be accomplished in any number of ways currently known or invented in the future, but, as an illustrative example, in certain embodiments this module 207 may be an optical system comprising an imaging and tracking system of the retina to locate the position of cells in realtime, and a scanned laser raster beam that is modulated as a function of position in order to deliver the desired micro-dose as it scans over a cell. In a specific exemplary embodiment this optical system may be, or may include, an adaptive optics scanning laser ophthalmoscope (further details are described below in this detailed description). In certain embodiments, a laser is scanned over the retina, and the intensity of the laser is varied as a function of spatial position, such that, when it is over a photosensitive cell with a specific target stimulus, the intensity of the laser is such that an exposure is delivered to the cell taking into account the wavelength of the laser and the photo-responsivity of the cell to light of that wavelength. In a specific exemplary embodiment, a single laser of a fixed wavelength is scanned, and the intensity of the laser is modulated in proportion to (a) the desired stimulus at a cell, and (b) the photo-responsivity of the cell in question to light at the laser's frequency.

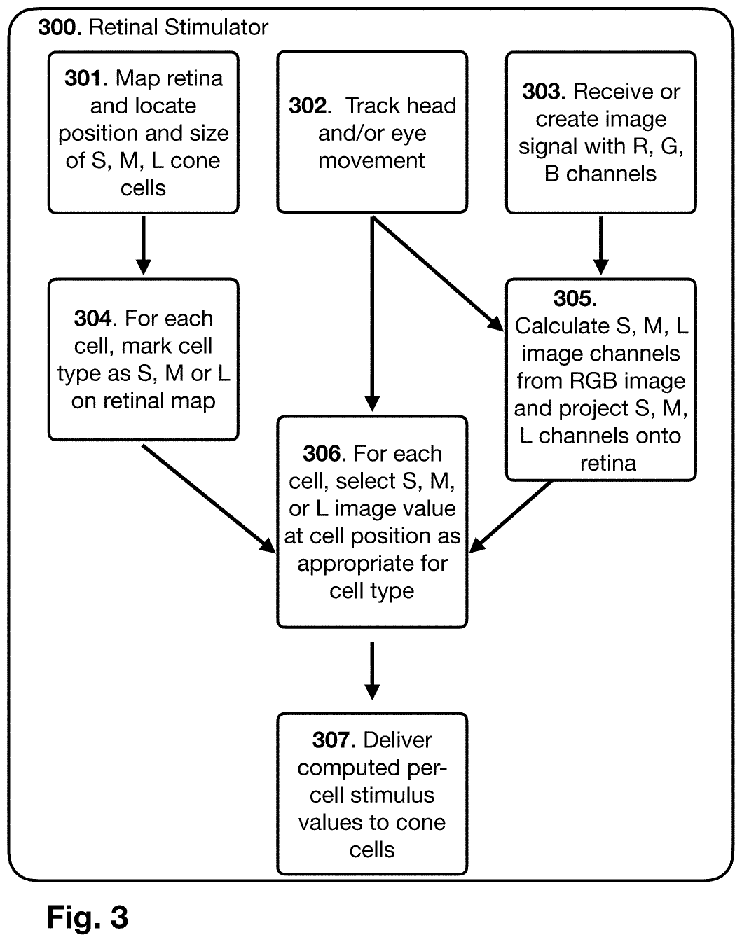

In another specific exemplary embodiment, a color image is displayed for the user. The following description refers to FIG. 3 in describing a specific exemplary implementation of this embodiment of the present invention in the context of a Retinal Stimulator 300. One module 301 of the architecture maps the retina and geometry (e.g. location, shape and/or size) of the various S, M and L cone cells. While this may be accomplished in any number of ways currently known or invented in the future, certain exemplary embodiments may use an adaptive optics scanning and tracking laser ophthalmoscope to image the retina, as described above, at a level that permits individual retinal cells to be discerned. Another module 304 then marks each cell type (e.g. S, M or L) on the retinal map. Another module 303 receives or creates a desired RGB image signal to be displayed to the user. In a manner similar to that already described above, module 302 tracks the head and/or eye movements of the user, which may vary as a function of time, such that the position and gazing direction of the eye relative to the world may be determined. Another module 305 accepts the head and/or eye tracking geometry and the desired RGB image signal, computes an SML image composed of corresponding S, M and L color channels derived from the RGB data (as with standard color space transformations, such as described in Mark Fairchild's book titled “Color Appearance Models, 3rd Edition”, published by Wiley in 2013 and incorporated here, in its entirety, by reference), and further computes the transformation and/or projection of the desired SML image signal onto the retinal map. Accordingly, module 306 computes target stimulus value for various S, M and L cone cells in the retinal map by selecting and/or sampling and/or estimating the value of the corresponding channel at the position in the desired SML image signal that corresponds to the specific cone cell in question. Module 307 delivers the desired per-cell stimulus to the various S, M and L cone cells in the retina, using techniques equal, similar or equivalent to those already described above.

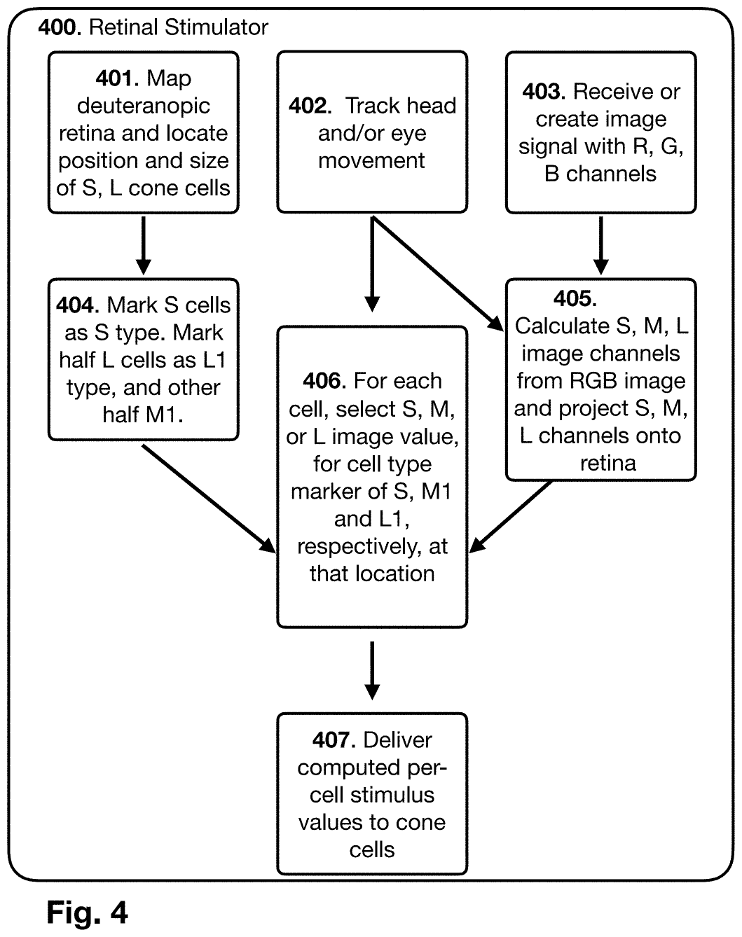

Referring now to FIG. 4, yet another specific exemplary embodiment of the invention in the context of a Retinal Stimulator 400 is directed at treating color blindness in an individual. Let us assume for the present discussion, without loss of generality, that the color blind user is a deuteranope (missing M-type cone cells). The idea is to inject the M-type stimulus values into a spatially distributed portion of the cells of one of the cone types that the user does possess. One module 401, using methods similar or equivalent to those already described above, maps the retina and geometry (e.g. location, shape and/or size) of the various S and L cone cells that the user possesses (no M cone cells are present to map in this exemplary application). Another module 404 then marks, in this example, the S cells as type S, half the L cells as type L1 and other half as type M1. Another module 403 receives or creates a desired RGB image signal to be displayed to the user. In a manner similar to that already described above, module 402 tracks the head and/or eye movements of the user, which may vary as a function of time, such that the position and gazing direction of the eye relative to the world may be determined. Another module 405 accepts the head and/or eye tracking geometry and the desired RGB image signal, computes an SML image composed of corresponding S, M and L color channels derived from the RGB data (as with standard color space transformations, such as described in Mark Fairchild's book titled “Color Appearance Models, 3rd Edition”, published by Wiley in 2013 and incorporated by reference above), and further computes the transformation and/or projection of the desired SML image signal onto the retinal map. Accordingly, module 406 computes target stimulus value for the various cone cells in the retinal map as follows: considering the SML triplet of values at the location over the cone cell in question, if the cell is type S, select the S value of the triplet; if the cell is type L1, select the L value of the triplet; and if the cell is type M1, select the M value of the triplet. The effect of this is to inject the M-type imagery into the half of the user's L cone cells that have been labeled type M1. Module 407 delivers the desired per-cell stimulus values to the various cells in the retina, using techniques equal, similar or equivalent to those already described above. In contrast to natural viewing, in which the user's brain receives no M-type imagery of the world, in this exemplary embodiment the user does receive and communicate to the brain the M-type imagery of the scene. According to the application, the user may achieve trichromatic color functionality, and/or perceive trichromatic color vision in spite of being a dichromatic color-blind individual. With regards to the exemplary embodiment drawn in FIG. 4 and described in detail here, one skilled in the art will recognize that: various modifications and alternatives may be employed to target users of different types of color blindness, including missing different types of cone cells, where the image signals corresponding to the missing or anomalous color channels are injected into chosen subsets of the remaining color channels; that variants of these embodiments may apply in which the user does not lack M-type cone cells (for example) but that they are anomalous with spectral photoresponse shifted toward that of L-type cones, and that in this case the M and L type cone cells could be effectively considered a single set of L-type cones with respect to the description of the embodiment above.

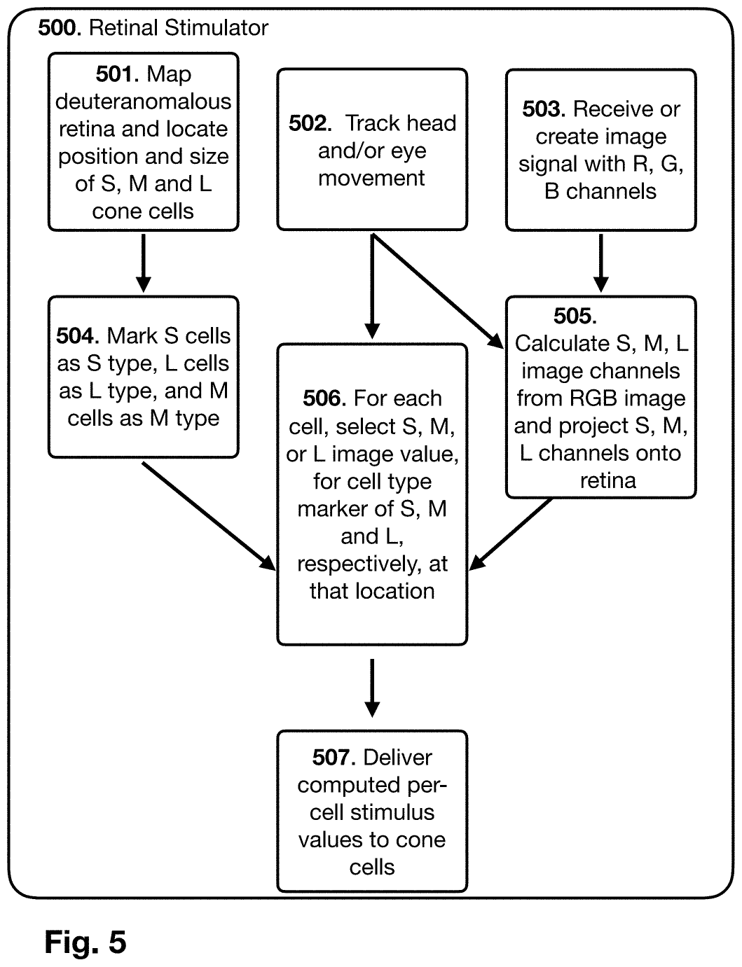

Referring now to FIG. 5, yet another specific exemplary embodiment of the invention in the context of a Retinal Stimulator 500 is directed at treating color blindness of the type associated with having an anomalous photopigment resulting, for example, in M and L cone cells that are more similar in spectra photoresponse than usual. Let us assume for the present discussion, without loss of generality, that the color blind user has deuteranomaly (M-type cone cells have spectral response shifted towards that of L-type cones). The idea is to calculate and inject the correct M-type stimulus values into the anomalous M cells that the user possesses. One module 501, using methods similar or equivalent to those already described above, maps the retina and geometry (e.g. location, shape and/or size) of the various S, anomalous M and L cone cells that the user possesses. Another module 504 then marks, in this example, the S cells as type S, the L cells as type L and the anomalous M cells as type M. Another module 503 receives or creates a desired RGB image signal to be displayed to the user. In a manner similar to that already described above, module 502 tracks the head and/or eye movements of the user, which may vary as a function of time, such that the position and gazing direction of the eye relative to the world may be determined. Another module 505 accepts the head and/or eye tracking geometry and the desired RGB image signal, computes an SML image composed of corresponding S, M and L color channels derived from the RGB data (as with standard color space transformations, such as described in Mark Fairchild's book titled “Color Appearance Models, 3rd Edition”, published by Wiley in 2013 and incorporated by reference above), and further computes the transformation and/or projection of the desired SML image signal onto the retinal map. Accordingly, module 506 computes target stimulus value for the various cone cells in the retinal map as follows. considering the SML triplet of values at the location over the cone cell in question, if the cell is type S, select the S value of the triplet; if the cell is type L, select the L value of the triplet; and if the cell is type anomalous M, select the M value of the triplet. The effect of this is to inject the correct M-type imagery values into the user's anomalous M-type cone cells. Module 507 delivers the desired per-cell stimulus values to the various cells in the retina, using techniques equal, similar or equivalent to those already described above. In contrast to natural viewing, in which the user's brain receives little to none of the correct M-type imagery of the world, in this exemplary embodiment the user does receive and communicate to the brain the correct M-type imagery of the scene. According to the application, the user may achieve trichromatic color functionality, and/or perceive trichromatic color vision in spite of being a color-blind individual. With regards to the exemplary embodiment drawn in FIG. 5 and described in detail here, one skilled in the art will recognize that various modifications and alternatives may be employed to target users of different types of anomalous color blindness, including those with tritanomaly or protanomaly.

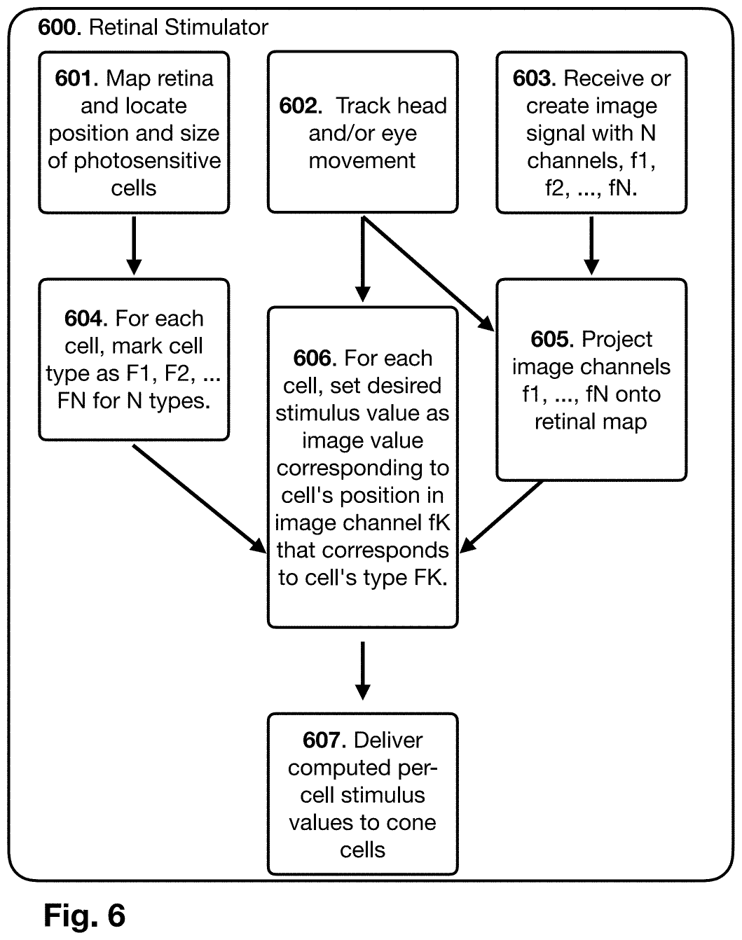

Various exemplary embodiments of the invention are directed at injecting into the retina and brain N-dimensional information about the spectrum at each point in an image desired for display. Referring now to the Retinal Stimulator 600 of FIG. 6, an implementation of an exemplary embodiment of the present invention is described that is directed to this application. One module 601, using methods similar or equivalent to those already described above, maps the retina and geometry (e.g. location, shape and/or size) of the various photosensitive cells in the retina. Another module 604 then marks various cells as one of N virtual types, with corresponding labels F1, F2, . . . , FN. This subdivision of cells into groupings of this kind can be done in any arbitrary manner to suit the needs of the application. In some applications, it may be desirable for the subsets of cells with a single virtual label to be distributed in an approximately even manner across a portion of the retina. Another module 603 receives or creates a desired image signal with N channels to be displayed to the user. For example, we may consider the N image channels as represented by functions ƒ1(x, y), ƒ2(x, y) . . . ƒN(x, y), where (x, y) are the 2D coordinates within the image. Again, the creation of these N channels of images may be created in an arbitrary manner to suit the needs of the application, and certain exemplary embodiments of this aspect of the present invention are described in more detail below in relation to later figures. In a manner similar to that already described above, module 602 tracks the head and/or eye movements of the user, which may vary as a function of time, such that the position and gazing direction of the eye relative to the world may be determined. Another module 605 accepts the head and/or eye tracking geometry and the desired N-channel image signal, and computes the transformation and/or projection of the desired image signal onto the retinal map. Let us denote the transformed image channels ƒ1^{t}(x,y), ƒ2^{t}(x,y) . . . ƒN^{t}(x,y) Accordingly, module 606 computes target stimulus values for the various photosensitive cells in the retinal map as follows: if the cell in question is of virtual type FK (where K is an integer in the range [1,N]) and the cell is located in the transformed image at location (x₀, y₀), then the stimulus value for the cell is chosen as ƒK^{t}(x₀, y₀). In words, the stimulus value for the cell is chosen as the value of the image channel that corresponds to that cell's virtual type, and at the location coinciding with the cell after the image channel is transformed and projected onto the retina. Module 607 delivers the desired per-cell stimulus values to the various cells in the retina, using techniques equal, similar or equivalent to those already described above. In contrast to natural viewing, in which the user's brain receives three dimensional information about the spectral information at points in the image, this exemplary embodiment delivers N-dimensional information.

Various embodiments of the present invention make use of the ability to stimulate patches of cone cells on the retina with any desired ratio of relative intensities (S,M,L) that are not subject to the constraints in Equations 1-3. Let us now refer to the SML chromaticity diagram in FIG. 8, where normalized S+M+L=1 for all points inside the triangle. A point 801 inside the natural human color gamut may be chosen, its (S,M,L) points determined by the barycentric coordinates of the point within the triangle, and these (S,M,L) values scaled by a chosen luminance to determine per-cone stimulus values in the region of an image corresponding to this desired color. These values will cause the user to perceive the corresponding natural color. Alternatively, a point 802 outside the natural human color gamut maybe chosen, and per-cell stimulus values can be computed with exactly the same procedure. These values may cause the user to perceive colors outside the natural human color gamut. The desired per-cell values could be computed at a per-cell stimulus calculation module (e.g. 106 in FIG. 1) and physically delivered to the cell by a per-cell stimulus delivery module (e.g. 107 in FIG. 1), as in descriptions of various other embodiments elsewhere in this document.

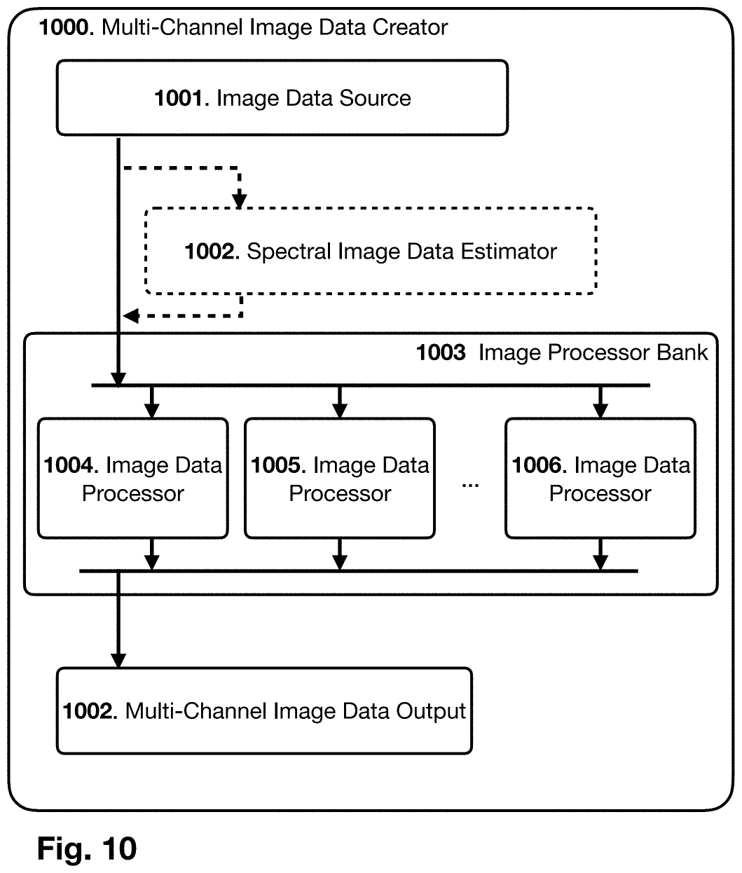

Referring now to FIG. 10, the figure depicts an exemplary implementation of the Image Data Creator 103 according to certain embodiments of the present invention. Here, a Multi-Channel Image Data Creator 1000 outputs image data 1002 with multiple channels. This Creator 103 begins with an Image Data Source 1001 that is optionally passed through a Spectral Image Data Estimator 1002 that may estimate full spectral information at each position of the image. The imagery is passed into an Image Processor Bank 1003 that contains N Image Data Processors, depicted as 1004-1006. These Image Data Processors each receive the imagery and computes a processed image from it. The image outputs from these N Processors are combined into the multi-channel image output 1002.

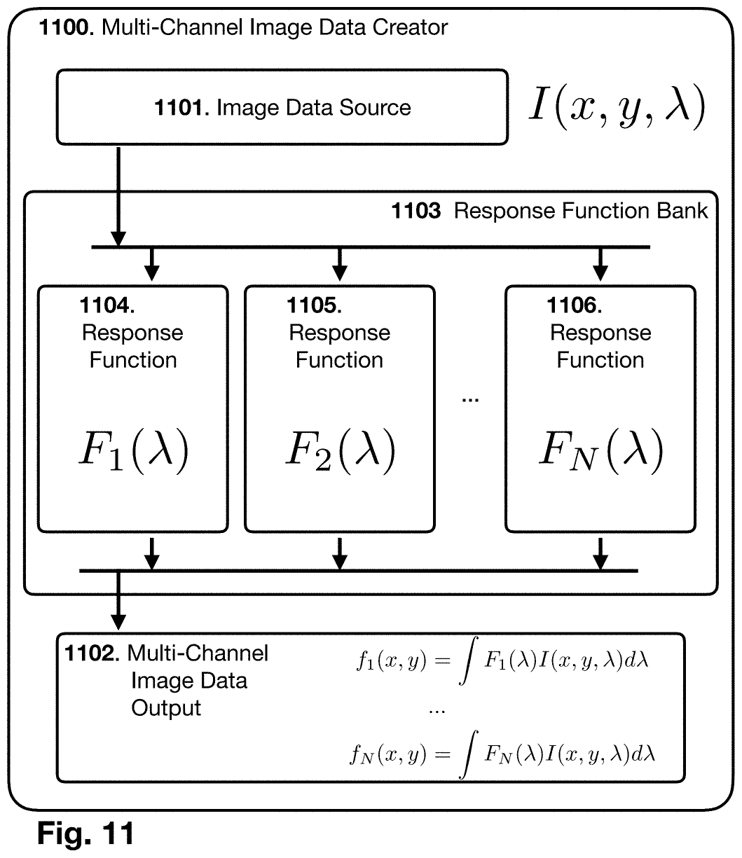

Referring now to FIG. 11, the figure depicts another exemplary implementation of the Image Data Creator 103 according to certain embodiments of the present invention. Here, a Multi-Channel Image Data Creator 1100 outputs image data 1002 with multiple channels. This Creator 103 begins with an Image Data Source 1101 that represents, estimates or is equivalent to a function I(x, y, λ) of spatial image position (x, y) and auxiliary variable(s) λ. In certain specific exemplary embodiments, I also vary as a function of time or other variables, which is normal even though not explicitly stated. This imagery I is passed into a Response Function 1103 that contains a plurality of, say, N, Response Functions 1104-1106, which contain functions Fᵢ(λ), where i runs from 1 to N. The Creator then computes the projection of the image I onto the response functions, to generate N output values that define a multi-channel image output 1102. In particular, the i'th channel of the image is given by, estimated by, or equivalent to a function

ƒᵢ(x,y)=^{[}Fᵢ(λ)I(x,y,λ)dλ.

In certain specific exemplary embodiments of this Creator 1000, the auxiliary variable λ is the wavelength of light; the function I(x, y, λ) is a hyperspectral image; the functions Fᵢ are spectral response functions for N classes of virtual photoreceptor types; and the ƒᵢ(x, y) are the images corresponding to the stimulus values for virtual photoreceptors of the corresponding type in response to incoming light defined by I(x, y, λ). The virtual photoreceptor spectral response functions can be any positive function (or indeed negative, as long as the resulting ƒᵢ(x, y) functions are clamped to positive functions) of A, and there can be any number of these response functions. The number and shape of the response functions depend on the application, and all number and function types are intended within the scope and spirit of certain embodiments of the present invention. Now let us turn to a number of specific exemplary embodiments of such virtual photoreceptor response functions to further understand the scope and potential of this approach.

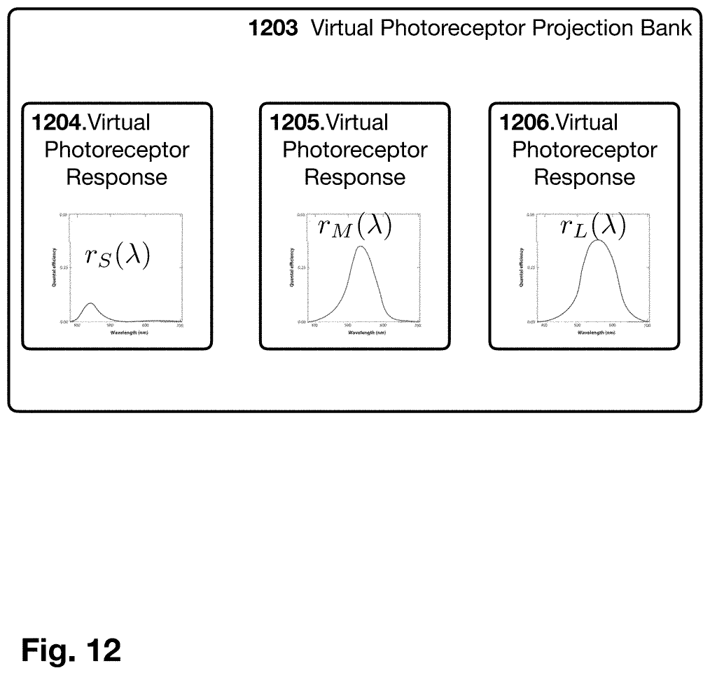

Referring now to FIG. 12, the figure depicts a Virtual Photoreceptor Projection Bank 1203 that is an exemplary embodiment of the Response Function Bank 1103. In this case, the bank contains three virtual photoreceptor spectral response curves 1204,1205,1206, which are chosen to be equivalent to the spectral response functions for the human S, M and L cones—respectively, r_{S}(λ), r_{M}(λ) and r_{L}(λ). The output of the Image Data Creator 1100 is therefore a multi-channel image representing the normal-color-vision response of the human retina to a particular scene.

In a specific exemplary embodiment, this Bank 1203 is used within a Multi-Channel Image Data Creator 1100 that is used to implement 603 within Retinal Stimulator 600; further, 604 have three types, with real S cells marked F1, real M cells marked F2 and real L cells marked F3. In this scenario, the Retinal Stimulator 600 is directed to creating a visual percept that has color equivalent to what would have been seen when viewing the scene under normal conditions (e.g. in the real world), and the addressable color gamut of this embodiment of the Retinal Stimulator 600 spans the full gamut of human color. This is unusual in that full color can be achieved even in specific embodiments where a single monochromatic laser (usually limited to producing percepts of a single hue) is used to stimulate cone cells in 607.

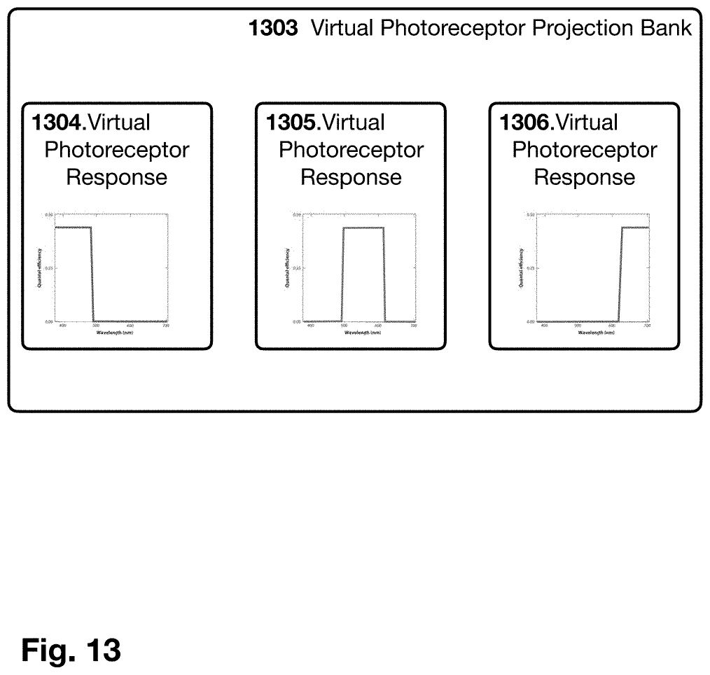

One limitation of certain embodiments using the Bank 1203 is that creating percepts of extended color gamut beyond the normal human gamut may not be possible. This is because the design of the photoreceptor response functions is such that resulting per-cone stimulus values will fall within stimulus ratios for S, M and L that follow the constraints of Equations 1-3 (e.g. it is not possible to obtain (S,M,L)=(0,1,0), even if normalized). In light of this, certain other exemplary embodiments of the present invention may implement the Response Function Bank 1103 in a manner that is directed, in part, to allowing targeting of all possible triplets of S, M and L. An example of such a bank is depicted in FIG. 13. This Bank 1303 contains three virtual photoreceptor response functions 1304,1305,1306 (which may be used as described above in embodiments whereby the resulting image channels are delivered to, respectively, S, M and L cone cells in the retina). Here, the exemplary response functions 1304,1305,1306 have the property that each contain wavelengths where it is non-zero, but the other response functions are equivalent to zero. This characteristic allows targeting of all possible triplets of S, M and L, and to address extended color gamut.

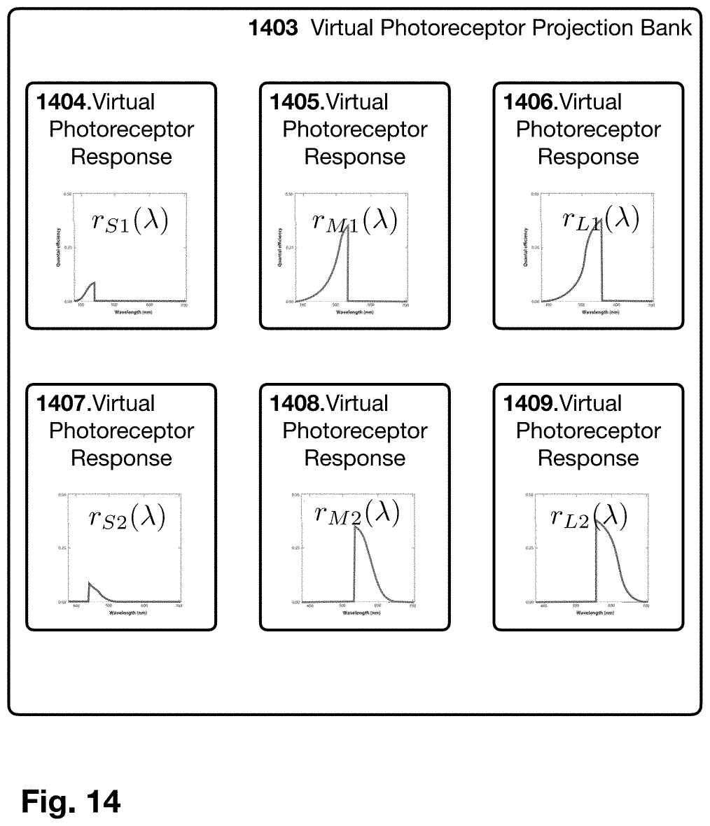

In general the Response Function Bank 1203 may utilize an arbitrary numbers of response functions. Referring now to FIG. 14, as an example of a larger number of response functions, we see a specific exemplary embodiment of a Bank 1403 with six photoreceptor types. These six photoreceptor response functions can be arbitrary functions as described above, and the specific functions depicted in FIG. 14 are meant only as examples for concrete discussion to facilitate this discussion. Thus, one example of a set of six photoreceptor types 1404-1409: r_{S1}(λ), r_{M1}(λ), r_{L1}(λ), r_{S2}(λ), r_{M2}(λ) and r_{L2}(λ), as depicted in FIG. 14. In this case, r_{S1} and r_{S2} functions are chosen as the left and right halves of r_{S}, the S cone cell response function; and similarly for r_{M1} and r_{M2} relative to r_{M}, and r_{L1} and r_{L2} relative to r_{L}. Another exemplary set of photoreceptor functions could be a set of six non-overlapping or minimally-overlapping box functions that collectively span a range of wavelengths.

In certain embodiments, specific embodiments of this type of Bank 1403 may used in concert with the creation of retinal parameter maps (e.g. in 104) and procedures to compute per-cell stimulus values (e.g. in 106), as further described below, in order to inject six-dimensional spectral information into the retina and brain.

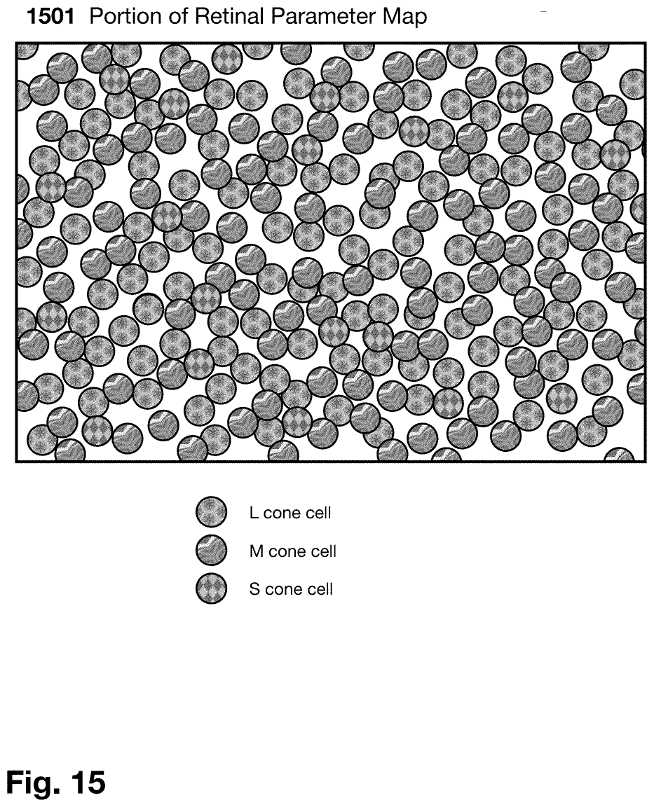

Referring now to FIG. 15, we see a depiction of a portion of Retinal Parameter Map 1501. While Retinal Parameter Maps can in general contain any type of spatially-varying data over the retinal surface, this figure depicts a distribution of retinal cone cells. In various embodiments of the present invention this information may be obtained by a retinal mapping step, such as in embodiments of Retinal Map 101, and stored in a data structure representing the locations (and optionally the size and shape) of various cones on the retina.

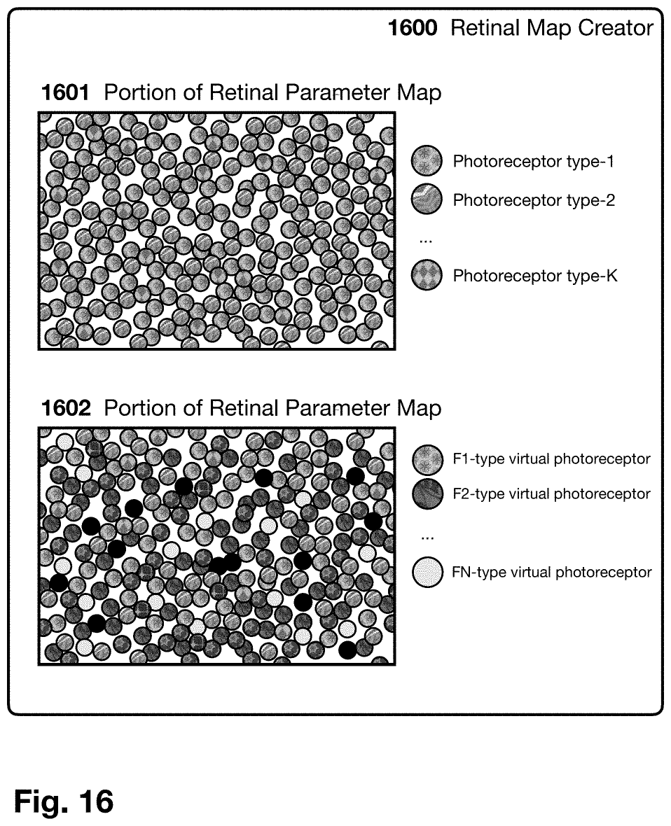

FIG. 16 depicts an exemplary embodiment of a Retinal Map Creator 1600. This Creator 1600 may be used in certain embodiments of the present invention as an implementation of 108 in a Retinal Stimulator 100. According to FIG. 16, the Retinal Map Creator may contain or receive a portion or a retinal parameter map 1601 that contains the location (and possibly the size and shape) of K classes of photosensitive cell types. In certain embodiments, these could include the S, M and L cone cells. In other embodiments, these may include rod cells, certain retinal ganglion cells, and/or any other class of real photoreceptor cell that is now known or discovered in the future. The Creator 1600 then derives, computes or assigns the location (and possibly the size and shape) of N classes of virtual photoreceptor types. In certain embodiments these virtual photoreceptor types may include real S, M and L cone cells, rods, photosensitive retinal ganglion cells, or other photosensitive cells now known or discovered in the future. In other embodiments, these virtual photoreceptor types may include imaginary or engineered photoreceptor types, such as those corresponding to the exemplary spectral response functions depicted in FIG. 13 and FIG. 14. There are a multitude of choices for the number and type of the K classes and the N virtual classes, which depend on the application of specific embodiments of the present invention, and all such number and types are intended to fall within the scope and spirit of the present invention.

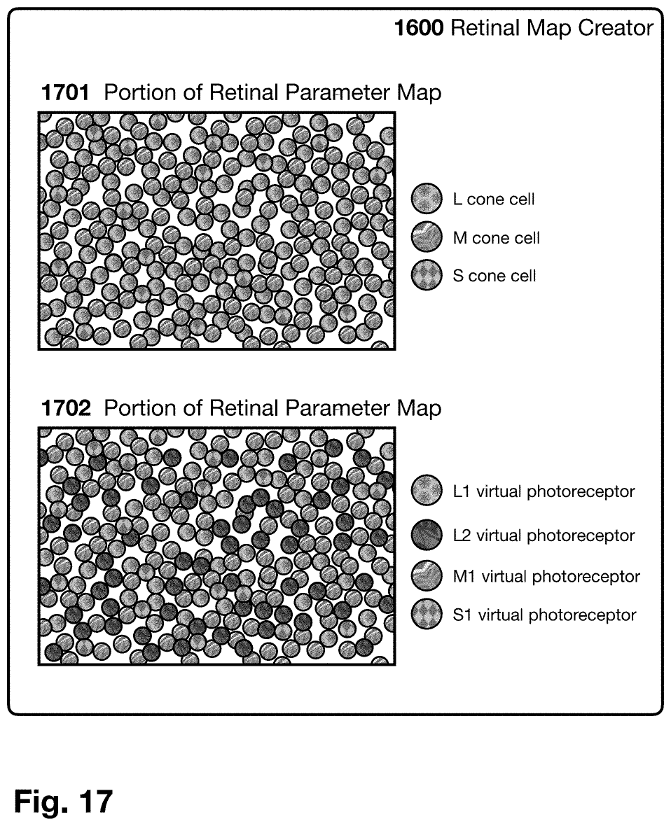

Referring now to FIG. 17, we see a specific exemplary embodiment of a Retinal Map Creator 1600, and particularly one strategy of the assignment of virtual photoreceptor types 1702 in relation to the map of real cone cells 1701. In this exemplary embodiment, the real locations of S, M and L cone cells 1701 are used to produce a map of virtual photoreceptor types L1, L2, M1 and S1, such that S cones are marked S1, M cones are marked M1, and L cones are split between types L1 and L2. In some embodiments, the distribution of L1 and L2 labels is chosen to be approximately spatially uniform. In certain embodiments of a Retinal Stimulator, this 4-channel distribution of labels of photoreceptor types may be used in concert with an embodiment of a Multi-Channel Image Data Creator 1100 in which the creator makes use of a response function bank 1103 of 4 photoreceptor functions, one each corresponding to virtual photoreceptor labels S1, M1, L1 and L2. In this embodiment, the values created by spectral projection onto these four virtual photoreceptor response functions are injected into the cells labeled accordingly. In a certain exemplary embodiment, the S1 label corresponds to a response function equivalent to r_{S}(λ), the S cone response: the M1 label corresponds to a response function equivalent to r_{M}(λ), the M cone response; the L1 label corresponds to a response function equivalent to r_{L}(λ), the L cone response; and the L2 label corresponds to a fourth, arbitrary response function. In this embodiment, the user may perceive colors in a four-dimensional space corresponding to tetrachromacy defined by these four described photoreceptor response functions.

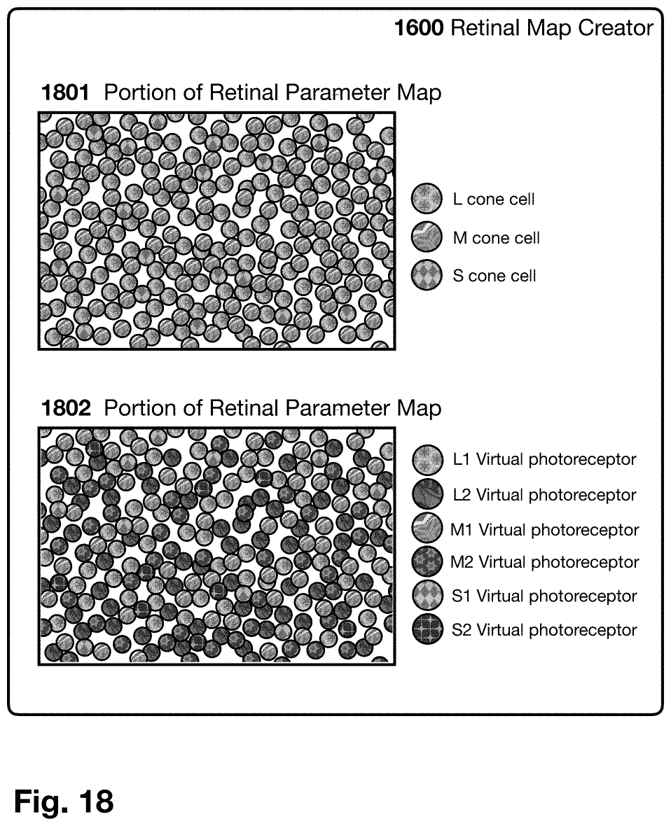

Referring now to FIG. 18, we see another specific exemplary embodiment of a Retinal Map Creator 1600, and another particular strategy of the assignment of virtual photoreceptor types 1802 in relation to the map of real cone cells 1801. In this exemplary embodiment, the real locations of S, M and L cone cells 1701 are used to produce a map of virtual photoreceptor types L1, L2, M1, M2, S1 and S2, such that S cones are split between S1 and S2, M cones are split between M1 and M2, and L cones are split between types L1 and L2. In some embodiments, the distributions of L1, L2, M1, M2, S1 and S2 labels are chosen to be approximately spatially uniform. In certain embodiments of a Retinal Stimulator, this 6-channel distribution of labels of photoreceptor types may be used in concert with an embodiment of a Multi-Channel Image Data Creator 1100 in which the creator makes use of a response function bank of 6 photoreceptor response functions (e.g. 1403), one each corresponding to each of the virtual photoreceptor labels S1, S2, M1, M2, L1 and L2. In this embodiment, the values created by spectral projection onto these six virtual photoreceptor response functions 1404-1409 are injected into the cells labeled accordingly. In this exemplary embodiment, the user may perceive colors in a six-dimensional space defined by the six photoreceptor response functions.

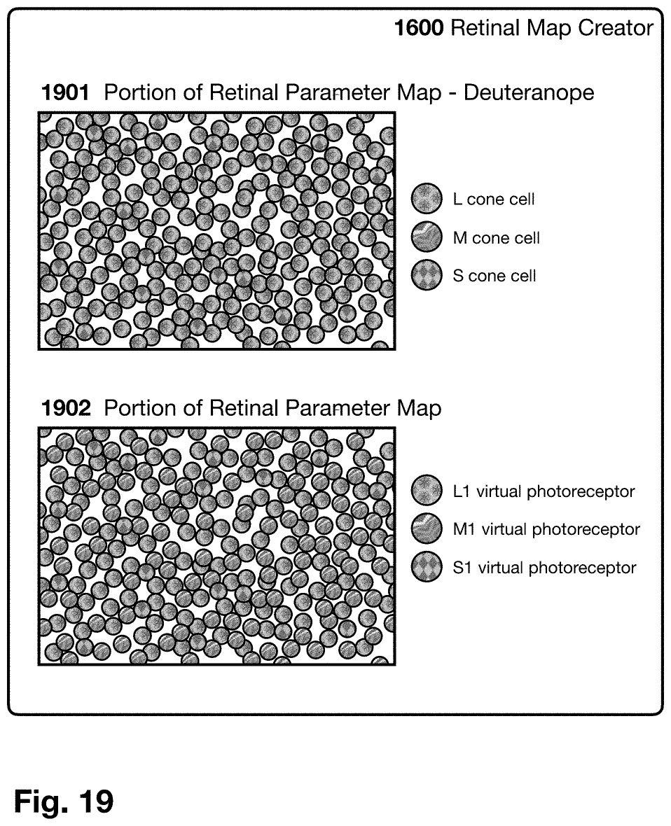

Referring now to FIG. 19, we see another specific exemplary embodiment of a Retinal Map Creator 1600, directed to creating trichromatic color functionality for an individual with color blindness. Without loss of generality, we consider here color blindness resulting from missing M cone cells, as depicted in the portion of the retinal parameter map 1901 depicting real cone cell locations (and possibly size and shape). In this exemplary embodiment, the creator assigns parameters to each of the cones to create virtual photoreceptor types 1902 equivalent to having all three S, M, L cones. In particular, the S cone cells are labeled type S1, and the L cone cells are split into two sub-populations labeled, respectively, L1 and M1. When used in embodiments of the present invention that, for example, compute S, M and L image values as described elsewhere in this detailed description, and inject those values into the cells here labeled, respectively, S1, M1 and L1, the viewer's retina is presented with trichromatic color information injected into the cells of the retina. In certain applications of this particular embodiment of the present invention, a dichromatic viewer may achieve trichromatic vision functionality. In certain applications of this particular embodiment of the present invention, a dichromatic viewer may perceive full trichromatic color vision, perceiving colors that could not be perceived naturally. As described above and elsewhere in this detailed description, one skilled in the art will recognize that the principles conveyed in the preceding description may be applied to trivially modify or permute the cell types in order to address color blindness involving missing L and/or S cells rather than M.

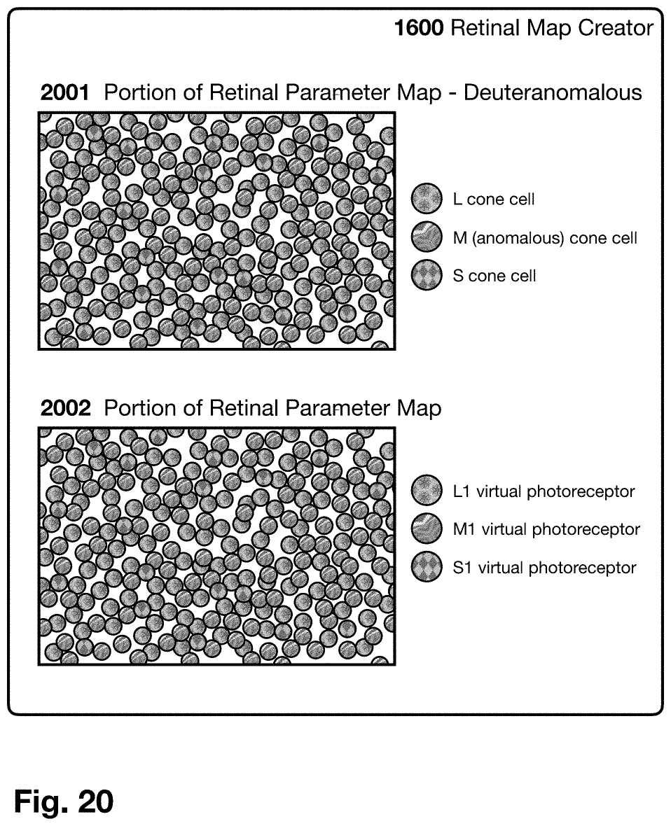

Referring now to FIG. 20, we see yet another specific exemplary embodiment of a Retinal Map Creator 1600, directed to creating trichromatic color functionality for an individual with color blindness of the anomalous variety, where one or some of the photoreceptor response functions are anomalously shifted to be closer to (less distinguishable) from one of the other response functions. Without loss of generality, we consider here anomalous color blindness resulting from anomalous M cone cells that have response shifted towards the L cone response. The resulting retinal parameter map of cone types 2001 may appear normal with three cone cell types. In this exemplary embodiment, the creator 1600 assigns parameters to each of the cones such that the S cone cells are labeled type S1, anomalous M cones are labeled type M1, and L cones are labeled L1. When used in embodiments of the present invention that, for example, compute (non-anomalous) S, M and L image values as described elsewhere in this detailed description, and inject those values into the cells here labeled, respectively, S1, M1 and L1, the viewer's retina is presented with normal trichromatic color information injected into the cells of the retina. In certain applications of this particular embodiment of the present invention, a deuteranomalous (M anomalous) viewer may achieve normal trichromatic vision functionality and perceive the world in full trichromatic color. As described above and elsewhere in this detailed description, one skilled in the art will recognize that the principles conveyed in the preceding description may be applied to trivially modify or permute the cell types in order to address color blindness involving anomalous L and/or S cells rather than M.

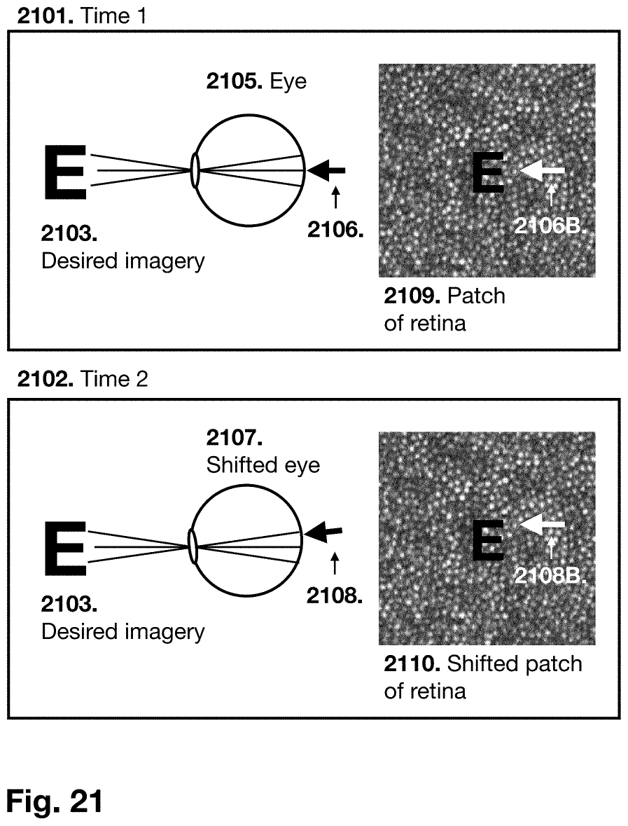

In various embodiments of the present invention, the functionality of the system depends on accurate alignment of the desired image on the retina, the current location of the retinal cell mosaic, and the delivery of computed stimulus values to various photosensitive cells. Referring now to FIG. 21, we see an example of how this alignment changes over time in an implementation of a specific exemplary embodiment of the present invention. Comparing time 1 2101 to time 2 2102, an example of a desired image is presented (a letter E 2103) that is the same between the two times. However, the eye 2105 viewing the image has shifted 2107 between time 1 and time 2. As a result, a point on the retina 2106 moves in time 2 relative to the projection of the image onto the retina. Similarly, patches of the retinal cell mosaic (2109 and 2110) are depicted with the same retinal cell 2106B shifting at time 2 to a new location 2108B relative to the image. According to this exemplary embodiment of the invention, one can see that since a point in the image may fall over a different retinal cell at different times, the stimulus value for various cells will vary with the movement of the eye relative to the scene. In general, the eye is constantly in motion relative to the scene, and even when a person carefully fixates on a seemingly stationary point, the cone mosaic is moving significantly, at the cellular level, relative to the projected image of the scene. It is described in various contexts elsewhere in this detailed description, how the computation of per-cell stimulus values depends on tracking the head and/or eye in order to compute the transformation of the desired image onto the retinal map, and the multitude of ways that the transformed image and retinal parameter maps may be used to compute per-cell stimuli.

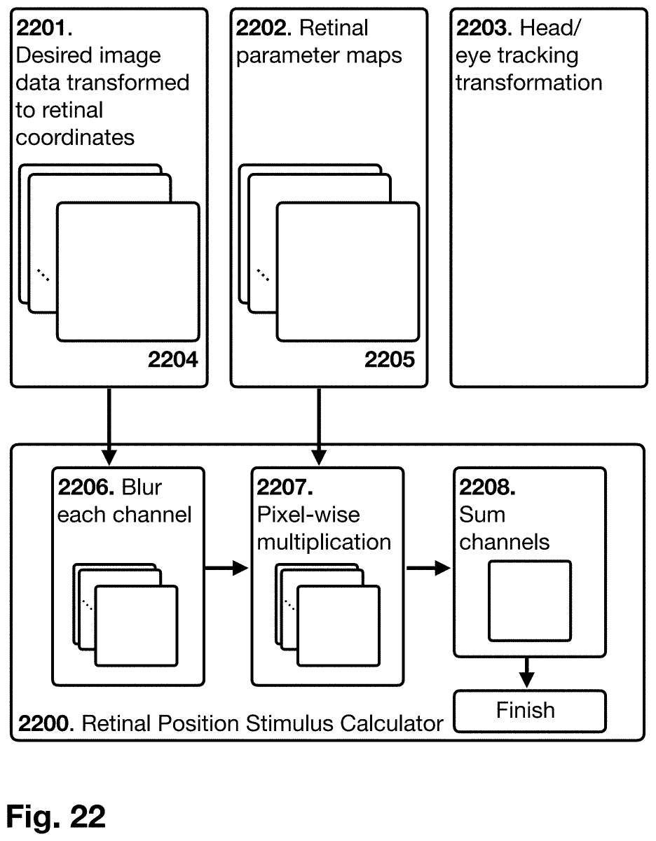

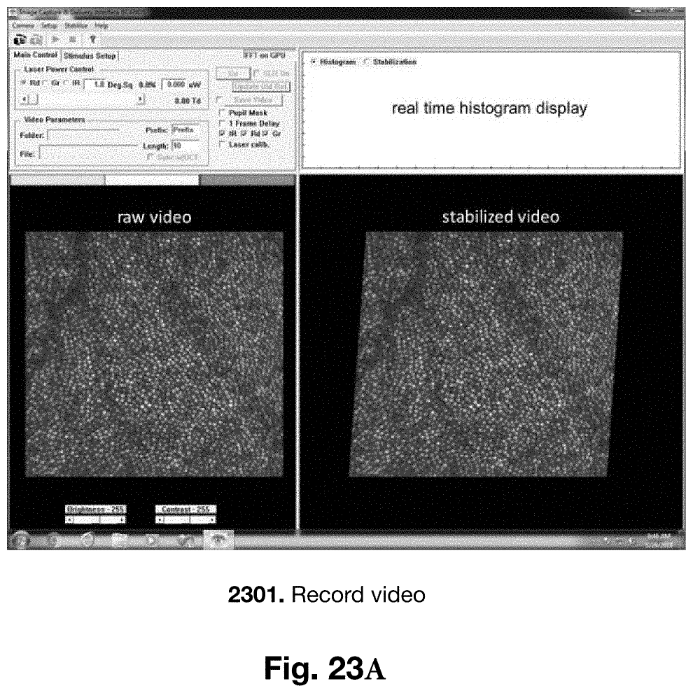

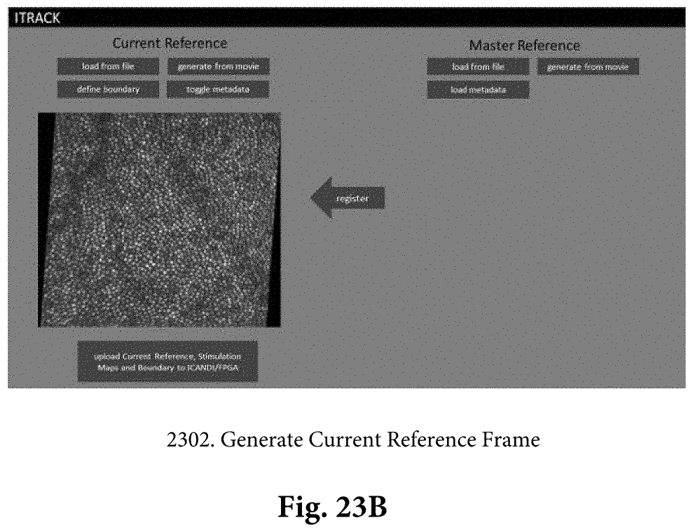

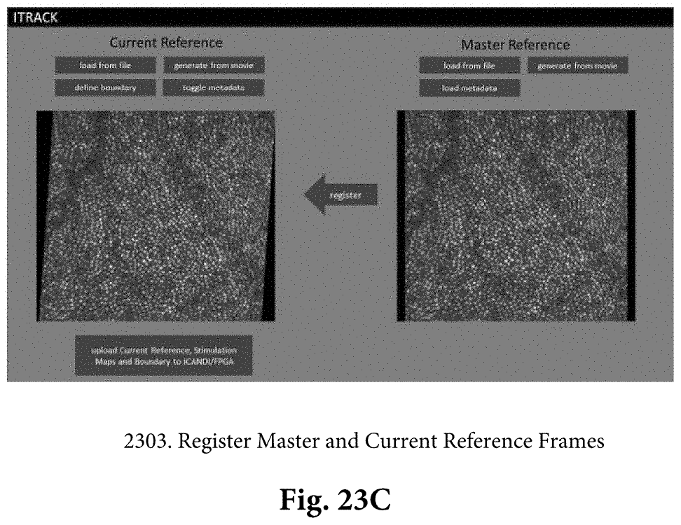

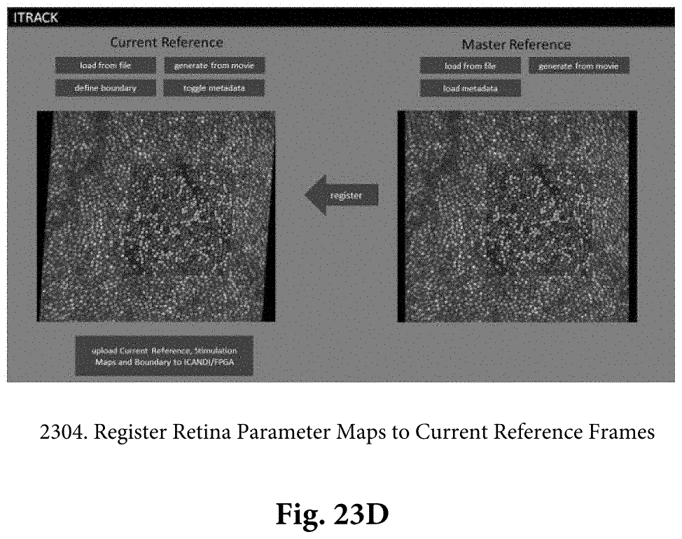

Referring now to FIG. 22, a Retinal Position Stimulus Calculator 2200 may be used in various embodiments of the present invention in order to calculate and facilitate delivery of desired per-cell stimulus values, as described here. The calculator receives information that may include the desired image transformed to retinal coordinates 2201, retinal parameter maps 2202 in retinal coordinates, and head/eye tracking information 2203 that relate imagery in the world to imagery projected on the retina. In certain embodiments of the present invention, the data and dataflow may be similar or equivalent to the following. The desired image data may be provided as multi-channel images 2204, each channel representing the continuous signal for a type of virtual photoreceptor; the retinal parameter maps may be comprised of multi-channel images 2205, where each channel relates to a single virtual photoreceptor type, and is zero everywhere except at one point over (and usually near the center of) each real retinal cell that has been assigned the corresponding virtual photoreceptor type; a scalar-valued image, representing stimulation levels at a raster of spatial positions on the retina, is computed by first blurring 2206 the desired image channels slightly (e.g. convolving them with a kernel approximately equal to the spatially varying sensitivity of the average cone cell), then multiplying 2207 each image channel pixel-wise with the corresponding channel of the retinal parameter maps and summing 2208 all the channels for each pixel. The net effect of this is to compute an estimate of the stimulus value at each light-stimulated raster position on the retina in order to deliver a stimulus onto each cell that corresponds to a weighted sum of the image values over the cell for the channel corresponding to that cell's virtual photoreceptor type.

Various embodiments of the present invention provide a method and/or system for delivering tristimulus SML values to a user's retina that spans a larger color gamut than normal vision. As described above, this is because various embodiments of the present invention allow any ratio of S, M and L values to be delivered to regions of the retina, freed from the normal constraints on the ratio defined by the spectral response functions of the three cone types and as defined by Equations 1-3. The ratios of S, M and L values that are not normal are referred to here as an extended color gamut. For example, a triplet of (S,M,L)=(0,1,0) of relative stimulus level is in the extended gamut, and certain embodiments of the present invention stimulate regions of the retina with this triplet, or other triplets in the extended color gamut, in order to produce percepts of colors beyond the natural human gamut. For certain embodiments, computing the desired SML triplet for cones to make use of the extended color gamut may utilize various maps of the new color space, including the extended gamut.

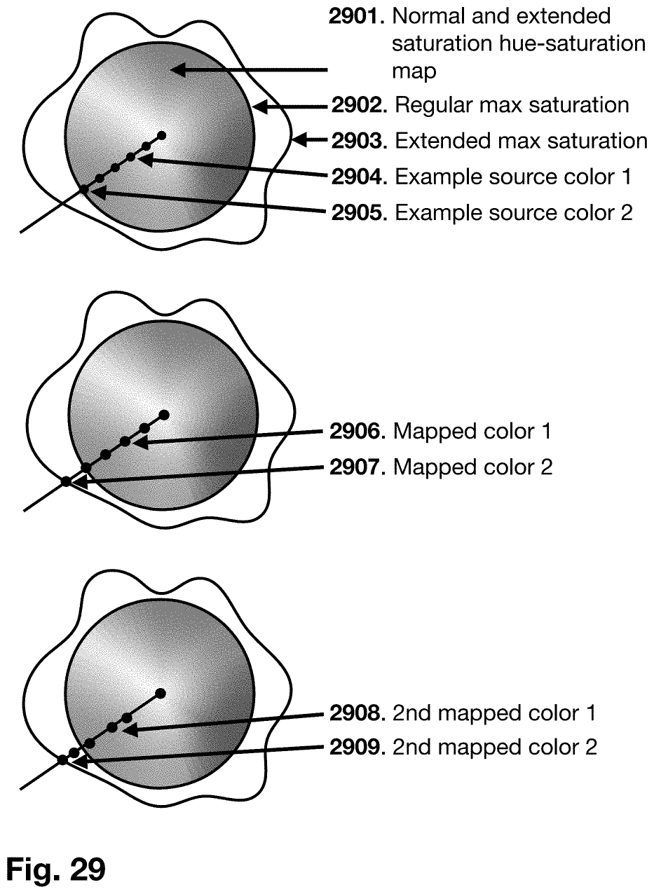

In certain embodiments of the present invention it may be desired to increase the saturation or colorfulness of the image without changing the hue, which may be called saturation stretching. FIG. 29 depicts a manner for implementing saturation stretching according to an exemplary embodiment of the present invention. 2901 depicts a hue-saturation map that contains colors in the normal human gamut as well as the extended color gamut addressable by various embodiments of the present invention and as described elsewhere in this detailed description. The map contains a disk that represents colors in the natural human gamut (similar to the hue/saturation portion of the common HSV color space), where the center of the circle is fully desaturated (white), and saturation increases towards the periphery of the disk 2902 where it attains a maximum for normal human color vision. In addition, the diagram depicts an illustration of the extended maximum saturation 2903 outside the periphery of the disk that represents colors that may be achieved with various embodiments of the present invention as described elsewhere in this detailed description. The hue of the color is constant along rays emanating from the center of the disk. In certain exemplary embodiments of the invention, points between the periphery of the disk and the boundary of the extended maximum saturation are determined by mapping perceptual measurements presented to human subjects with S,M,L values outside the natural human gamut and tasking them with matching the hue of the perceived color to a known color in natural human gamut (if possible), and ordering the saturation relative to known saturation levels in human gamut and other sampled S,M,L values in this color mapping procedure. As a byproduct of this mapping procedure, each point on the color space 2901 maps back to a an S,M,L stimulus value that perceptually mapped to that location on the color space. Given this normal and extended hue-saturation map, FIG. 29 depicts a series of colors of constant hue that span all saturation levels for normal human color vision. To extend the saturation for colors in, say, a regular RGB image with conventional color gamuts, for each pixel we can first determine its hue and saturation on the color space. For example, two such colors are source color 1 2904 and color 2 2905. Then, we can stretch the saturation of the source colors to higher values, potentially into the extended saturation portion of the space. 2906 and 2907 depict the increased-saturation colors corresponding to 2904 and 2905 after linear stretching of the saturation. 2908 and 2909 depict the increased-saturation colors corresponding to 2904 and 2905 after non-linear stretching of the saturation. After mapping, in order to display the saturation-stretched colors, the values are mapped to corresponding S,M,L triplets and delivered to the retina as described elsewhere in this detailed description according to other exemplary embodiments.

Various embodiments of the present invention use imaging and/or tracking and/or stabilization of retinal movement in order to determine the transformation and/or projection of desired imagery onto the retina and to determine the detailed geometric relationship of where this imagery falls onto the mosaic of photosensitive cells in the retina. Detailed descriptions of the imaging and tracking aspect of these exemplary embodiments are described further below in sections related to AOSLO, eye-tracking, ITRACK and RetCon.

Various embodiments of the present invention deliver the computed, desired stimulus value to various photosensitive cells in the retina by using laser stimulation possibly with stabilization of tracked retinal movement. Detailed descriptions of these stimulation and/or stabilization aspects of these exemplary embodiments are provided below, in sections related to AOSLO, eye-tracking, ITRACK and RetCon in the attached document.

In various embodiments of the present invention, portions of the embodiment may be implemented, as described in various contexts above, using systems that incorporate, or methods that utilize, an adaptive optics scanning laser opthalmoscope (AOSLO), combined with eye tracking and targeted stimulus delivery. Following portions of this detailed description describes additional detail on general implementation of these systems and methods, and specific details as relate to their adaptation or modification according to various exemplary embodiments of the present invention.

The following is a list of definition of terms used in following portions of this detailed description:

AOMControl—Matlab-based software for designing and running AOSLO experiments.

AOSACA—Adaptive Optics Sensing and Correction Algorithm. Custom software application for measuring and correcting the aberration of the eye.

AOSLO—adaptive optics scanning laser ophthalmoscope

Coretsumo—computation retinal supermosaicing. A procedure by which existing cones are stimulated to mimic a cone with different spectral sensitivity characteristics.

Current Reference Frame—high quality reference frame constructed in the current session. (A current reference might be better for tracking stability owing to variations in reflectivity of cones, torsion and small scaling changes over time)

Current Retinal ParameterMap—a corrected (scale, distortion, torsion) version of the Master Stimulation Map that corresponds to the Current Reference.

Fixed-Field Mode—a display where the boundaries of the stimulation frame are at a fixed location within the AOSLO raster field.

Stimulus Imagery—An N-layer image or video that is to be projected onto the retina.

Stimulus Frame—The boundary of the Stimulus Imagery to be delivered. The Frame may move in a manner that is contingent on retinal motion (i.e. gain of 1=stabilized)

ICANDI—Image capture and delivery interface (main AOSLO software).

ITRACK—software module within ICANDI that will enable more efficient experiments and improved reference frame generation.

ITRACKMaster Reference Frame—high quality reference frame constructed from a previous session.

Master Retinal ParameterMap—An N-layer specific stimulation pattern that is referenced to the ITRACK Master Reference Frame. (for example, three layers containing L, M and S cone locations).

ReCon—the complete system for retinally contingent display.

Stimulus Onto Retina Projection—The actual set of intensity values that will be delivered to the retina via modulation of the AOM (prior to scan distortion correction).

Various embodiments of the present invention utilize an Adaptive Optics Scanning Laser Ophthalmoscope: The AOSLO is a scanning laser ophthalmoscope, or SLO (Webb, Hughes, & Pomerantzeff, 1980, which is incorporated here in its entirety by reference) that uses adaptive optics, or AO (Liang, Williams, & Miller, 1997, which is incorporated here in its entirety by reference). The combination of AO and SLO was first demonstrated by Austin Roorda in a paper in 2002 (Roorda et al., 2002, which is incorporated here in its entirety by reference), and is also the subject of U.S. Pat. Nos. 6,890,076 and 7,118,216. An SLO records an image of the retina by recording the light scattered from a small focused spot that is scanned (typically in a raster pattern) across the retina. Each frame is recorded pixel-by-pixel and a computer is used to reconstruct and render each frame, or sequence of frames (i.e. video) to save or display on a monitor.

Certain AOSLO implementations are capable of recording videos of a human retina with a resolution of about 2 microns, sufficient to image individual cone and rod photoreceptor cells. Improvements in AOSLO system optical design since the original invention have led to improved resolution and contrast (Dubra et al., 2011; Merino, Duncan, Tiruveedhula, & Roorda, 2011, which is incorporated here in their entirety by reference).

Eye Tracking: Various embodiments of the present invention make use of eye-tracking techniques in which videos recorded from an SLO can be analyzed to track eye motion at rates that are higher than its frame rate (Mulligan, 1997; Sheehy, Arathorn, Yang, Tiruveedhula, & Roorda, 2012; Stetter, Sendtner, & Timberlake, 1996; which are both incorporated here in its entirety by reference). These analysis techniques applied to an AOSLO may be used to track eye motion on a cellular scale, as described in (Stevenson & Roorda, 2005; Vogel, Arathorn, Roorda, & Parker, 2006; which are incorporated here in its entirety by reference). Implementations of certain exemplary embodiments of the present invention utilize hardware and software to perform this high-speed, high accuracy tracking in real time according to the principles described in Arathorn et al., 2007; Yang, Arathorn, Tiruveedhula, Vogel, & Roorda, 2010; which are all incorporated here in their entirety by reference.

Targeted stimulus delivery: Various exemplary embodiments of the present invention make use of the raster-scanned beam in an SLO for imaging, and modulate the scanning beam to project images onto the retina. When the same laser is used to project a pattern on the retina, that pattern will also appear on the image that is recorded, as described in (Mainster, Timberlake, Webb, & Hughes, 1982; Timberlake, Mainster, Webb, Hughes, & Trempe, 1982; Webb & Hughes, 1981; which are incorporated here in their entirety by reference). When the same technique is applied in an AOSLO, it can deliver near diffraction-limited patterns onto the retina, since the AO corrects aberration of both in ingoing and outgoing light from the eye (Poonja, Patel, Henry, & Roorda, 2005, which is incorporated here in its entirety by reference). Various embodiments of the present invention combine this ability to deliver images to the retina with real-time eye tracking (see above) to project patterns to targeted locations on the retina (Arathorn et al., 2007; Tuten, Tiruveedhula, & Roorda, 2012; Yang et al., 2010; which are incorporated here in their entirety by reference). Further, specific exemplary embodiments use an AOSLO set up to scan multiple beams of different wavelengths, so that one wavelength (e.g. near infra-red) may be used for imaging and tracking, while one or more beams of a second wavelength (e.g. red and green) may be used to project a pattern to a targeted retinal location. To do this accurately, the transverse chromatic aberration of the eye is measured and corrected in certain exemplary embodiments of the present invention, according to the principles described in Harmening, Tiruveedhula, Roorda, & Sincich, 2012, which is incorporated here in its entirety by reference. Certain embodiments of the present invention utilize a fully equipped AOSLO system capable of tracking and measuring sensitivity thresholds of single cone photoreceptors, as described in Harmening, Tuten, Roorda, & Sincich, 2014, which is incorporated here in its entirety by reference.

System Operation: A particular implementation of the AOSLO-based system in certain exemplary embodiments of the present invention is controlled by several software modules. The adaptive optics system is run by the Adaptive Optics Sensing and Correction Algorithm (AOSACA). Imaging, tracking and stimulus delivery is run by the Image Capture and Delivery Interface (ICANDI), data input/output is run on a custom-written FPGA-based application, and vision testing experiments are run using a Matlab-based GUI interface called AOMControl. ITRACK and RetCon are integrated with these software modules, in these exemplary embodiments of the present invention.

Generation of Retinal Parameter Maps: According to certain exemplary embodiments of the current invention, Retinal Parameter Maps refer to any pattern that corresponds to specific retinal locations of any given individual. These maps may include, but are not limited to cone and or rod locations, cone spectral subtypes (L, M and S) or microvasculature. Cone and/or rod locations are determined by direct analysis of the scattered light images. Cones and rods appear as a mosaic of small, Gaussian-shaped spots in confocal AOSLO images. In certain exemplary embodiments, their locations are determined either manually, by semi-automated or fully automated methods (Cunefare et al., 2017; Li & Roorda, 2007; which are incorporated here in their entirety by reference).

In certain embodiments of the present invention, a map of cone spectral subtypes (L, M and S) for a specific individual to use the system is generated using high resolution retinal densitometry in a conventional flood illumination AO retinal camera (as described in Roorda & Williams, 1999, which is incorporated here in its entirety by reference) or in an AOSLO (as described in Sabesan, Hofer, & Roorda, 2015, which is incorporated here in its entirety by reference). Certain embodiments use more recent, and more efficient methods using phase-resolved optical coherence tomography. In general, those skilled in the art will recognize that the present invention may utilize any method now known or invented in the future for determining the types of cone cells, or any other photosensitive cell. Certain exemplary embodiments of the present invention determine a map of the microvasculalture using fluorescein angiography, optical coherence angiography (Braaf et al., 2011; Choi et al., 2013; which are incorporated here in their entirety by reference), or in an AOSLO directly using either phase-contrast (Sulai, Scoles, Harvey, & Dubra, 2014, which is incorporated here in its entirety by reference) or motion contrast (Tam, Martin, & Roorda, 2010, which is incorporated here in its entirety by reference) methods.

In certain exemplary embodiments of the present invention, a system called ITRACK may be used to aid in tracking the retina for determining the relative position of the eye to the world. In certain embodiments, a certain mode of operation for tracking and targeting stimulus delivery in ICANDI, a single AOSLO video frame is selected for use as a reference frame for real-time stabilization. Targets on the retina for stimulation (e.g. individual L, M or S cones, or retinal lesions) are identified only after the reference image from the subject/patient has been collected. For certain applications this may be inefficient and impose a bottleneck on our ability to perform functional testing in normal and diseased eyes. In contrast, certain exemplary embodiments of the present invention use a software module called ITRACK to serve several purposes. First, ITRACK may enable the generation of an improved reference frame. The reference frame (i) may be comprised of multiple frames and so will have higher signal to noise, (ii) may have reference frame distortions removed in software by dewarping the image (Bedggood & Metha, 2017; Stevenson & Roorda, 2005; Vogel et al., 2006; which reincorporated here in their entirety by reference), and (iii) may span a larger area in space and in pixels than a single frame reference. In these embodiments, ITRACK works together with the RetCon display.

Referring to FIG. 4. 23″-23&, the following describes exemplary operation of ITRACK in certain embodiments of the present invention:

1. ICANDI records a video 2301 of user's retina that contains a targeted retinal location.
2. ITRACK generates a high quality Current Reference Frame 2302 image of that location.
3. A registration between the ITRACK Master Reference Frame and the Current Reference Frame is computed 2303.
4. One or more Master Retinal Parameter Maps associated with the ITRACK Master Reference Frame are registered onto the Current Reference Frame 2304.
5. ICANDI computes a new set of Current Retinal Parameter Maps based on the registration parameters between the Master and Current Reference frame.
6. The Stimulus Imagery is loaded and positioned relative to the Current Retinal Map 2305. The Stimulus Imagery could be, for example, a static image, a simple colored square, or a movie. The Stimulus Imagery will define the boundaries of the Stimulus Frame, which may be smaller and fall within the bounds of the Current Retinal Parameter Maps.
7. ITRACK/ICANDI uploads the coordinates of the Stimulus Frame and the Current Retinal Parameter Maps to the FPGA board.

In certain exemplary embodiments of the present invention, RetCon Display may be used to implement various aspects of eye tracking, aligning retinal parameter maps and desired images, and computing per-cell or per-retinal-location stimulus values. In one embodiment of the present invention, when we place a stimulus on the retina, the entire stimulus pattern may be delivered. If it is stabilized relative to the retinal cone mosaic (gain=1) then the entire pattern moves along with the retina. A stimulus presented under these conditions will appear to move, then fade from view (Arathorn, Stevenson, Yang, Tiruveedhula, & Roorda, 2013, which is incorporated here in its entirety by reference).

The stabilized stimulus presents a viewing condition that is unnatural compared to normal human viewing of the world, and has been shown to hamper spatial vision (Ratnam, Domdei, Harmening, & Roorda, 2017, which is incorporated here in its entirety by reference). In another embodiment of the present invention, the movement of the image across the retina during normal fixation offers information to help the visual system disentangle the spatial and color variations in a scene. This may prevent the presented stimulus from fading from the user's perception, and to strengthen the chromaticity of the created visual percept. This exemplary mode of stimulus delivery and percept formation is called the RetCon display. In this exemplary embodiment of the invention, the implementation of this is called RetCon Mode and comprises the following steps:

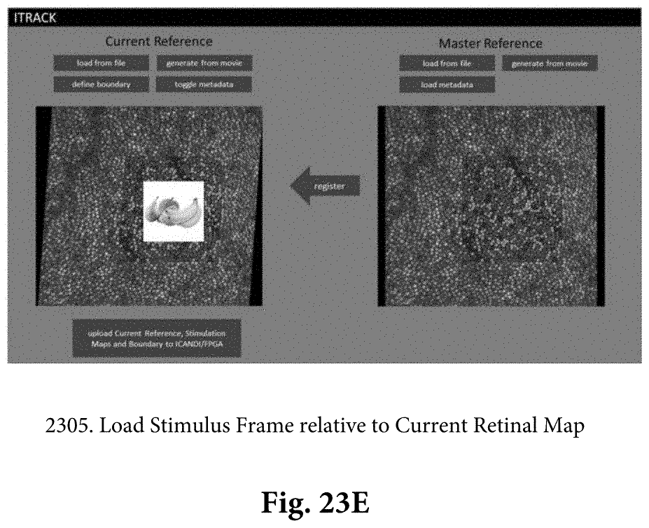

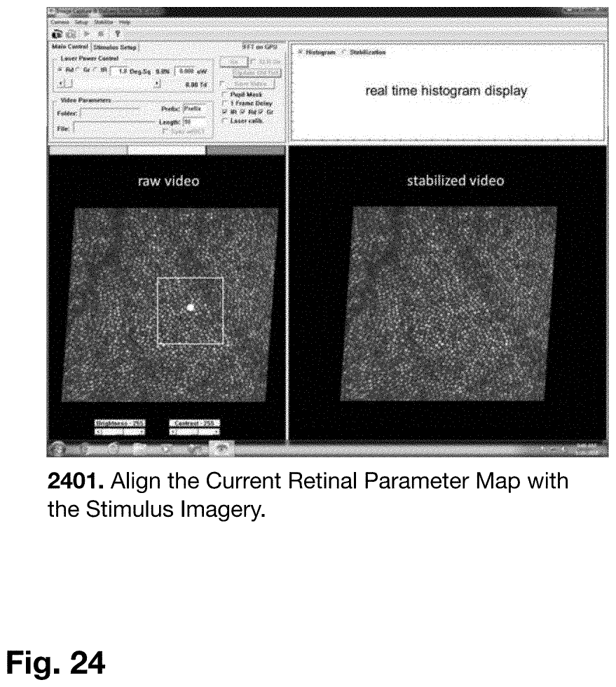

1. ICANDI uses the Current Reference Frame as the reference frame and displays a stabilized video relative to the Current Reference Frame.
2. The boundary of the Stimulus Frame may be indicated by digital marks on the Raw Video
3. The center of the Current Retinal Parameter Maps may also be indicated on the Raw Video with a digital mark, in order to allow gauging of tracking performance.
4. If desired, the geometric calibration can be adjusted (see 2401, referring to FIG. 24) between the fixation target of the user and the center of the Current Retinal Parameter Maps to achieve alignment near center of the Stimulus Frame.
5. In AOMControl the user may upload a unique Stimulus Imagery file (e.g. play a movie) and/or Current Retinal Parameter Map (e.g. to manipulate stimulation parameters) for each frame. As an explanatory example, the user may view a uniform green field and the Current Retinal Parameter Map may be switched from normal vision (e.g. LMS values of [0.5 1 0.5]) to an Oz Vision value outside the normal human color gamut (e.g. LMS values of [0 1 0])
6. In various embodiments, ICANDI may determine the retinal location just before the raster scans over the Stimulus Frame and will arm the AOM playout buffer with the Stimulus Onto Retina Projection, which is the sum of the product of the Stimulus Imagery Channels with the corresponding channels of the cropped section from the Current Retinal Parameter Map. This determination may be performed for the entire retinal field, or in certain embodiments it may be ideal to do so on a strip-by-strip, line-by-line or even pixel-by-pixel basis, subject to the communication and computational bandwidth of the available system.

Various concepts and applications of the RetCon display in exemplary embodiments of the present invention may be further understood by a few additional illustrative examples. Note that these exemplary embodiments are meant to reiterate and further clarify the concepts and potential applications of the present invention, and are in no way intended to fully enumerate all possible examples or imply any limits on the breadth or generality of the invention:

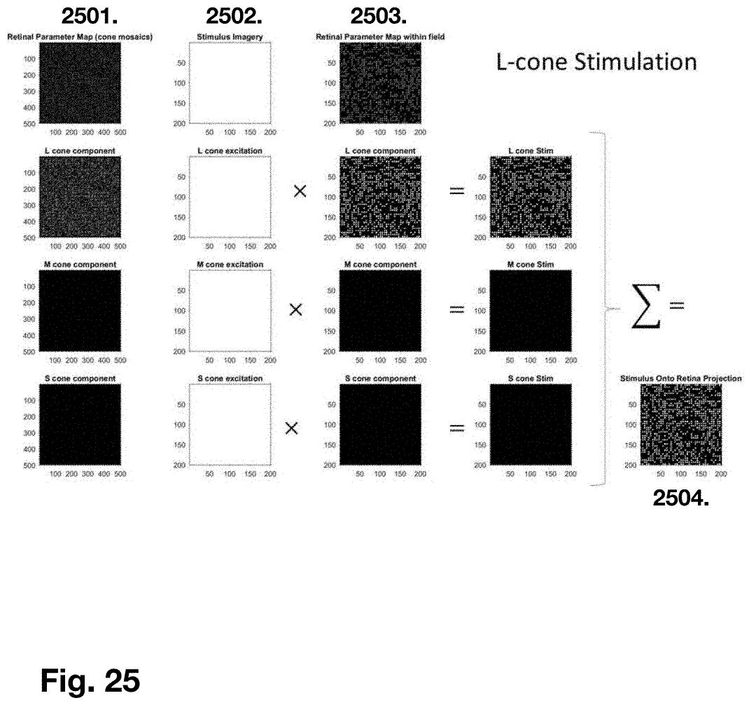

1. Referring to FIG. 25, certain exemplary embodiments of the present invention are directed to stimulate only L-cones within a square field that is fixed in world coordinates (i.e. appear fixed in the world and fixed in the AOSLO raster). The Stimulus Imagery 2502 would be a uniform square. The Stimulus Frame would be displayed with a gain of 0. The Current Retinal Parameter Map 2501 would be a map of the L-cones of the specific individual (read from the Master Retinal Parameter Map). As the eye moves, ICANDI reads the eye position and determines what part of the Current Retinal Parameter Map to read out. The product of the Stimulus Imagery 2502 and the Current Retinal Projection Map 2503 is the Stimulus Onto Retina Projection 2504, which gets played out onto the retina each frame.

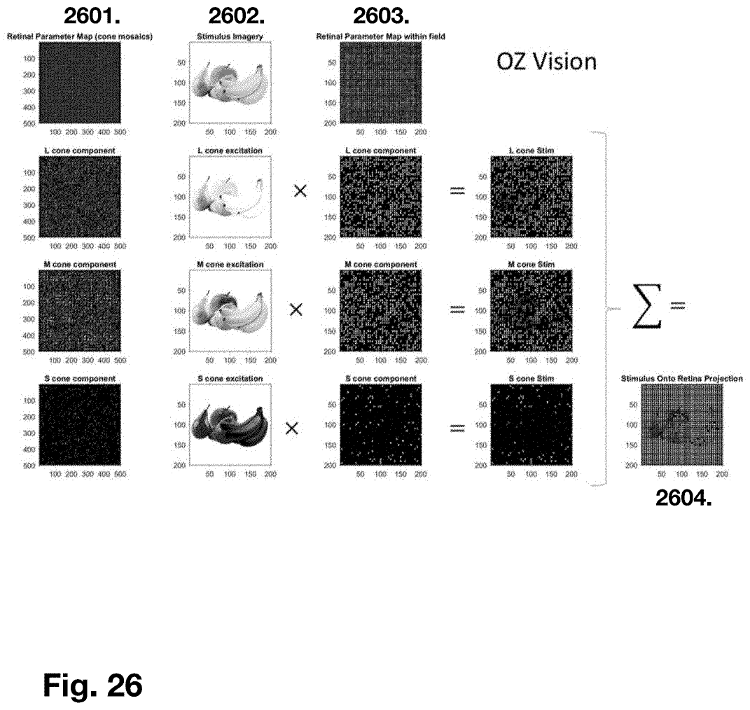

2. Referring to FIG. 26, certain exemplary embodiments of the present invention are directed to generate spatial metamer for a color image. The Stimulus Imagery 2602 would be a color image comprised of three layers, the L-component, the M-component and the S-component. The Stimulus Frame would be displayed with a gain of 0. The Current Parameter Map 2601 would be a three-channel image containing a stimulation pattern for the L-cones, the M-cones and the S-cones (read from the Master Retinal Parameter Map). As the eye moves, ICANDI reads the eye position and determines what part of the Current Retinal Parameter Map to read out. The product of the Stimulus Imagery 2602 and the Current Retinal Parameter Map 2603 generate three channels which are summed to generate the Stimulus Onto Retina Projection 2604, which gets played out onto the retina. In this scenario, a spatially modulated image from a single laser may be indistinguishable from the original color image.

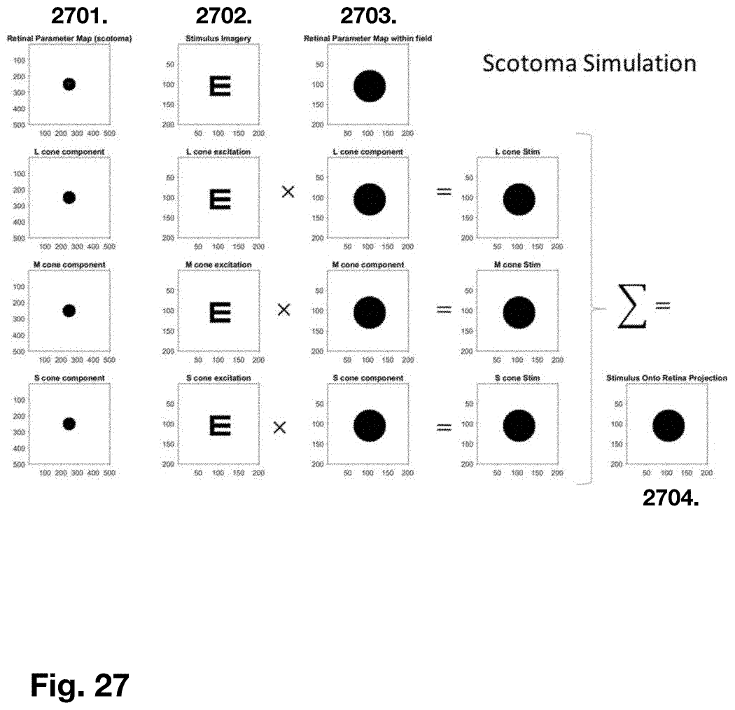

3. Referring to FIG. 27, certain exemplary embodiments of the present invention are directed to administer an acuity test with a simulated Scotoma. The Stimulus Imagery 2702 would be a letter ‘E’ (same in all layers). The Stimulus Frame would be displayed with gain of 0. The Current Parameter Map 2701 would be a dark circular patch of the size of the simulated scotoma (read from the Master Retinal Parameter Map). As the eye moves, ICANDI reads the eye position and determines what part of the Current Retinal Parameter Map to read out. The product of the Stimulus Imagery 2702 and the Current Retinal Projection Map 2703 is the Stimulus Onto Retina Projection 2704, which gets played out onto the retina each frame. In effect the E is fixed in world-coordinates and the scotoma travels with the eye motion.

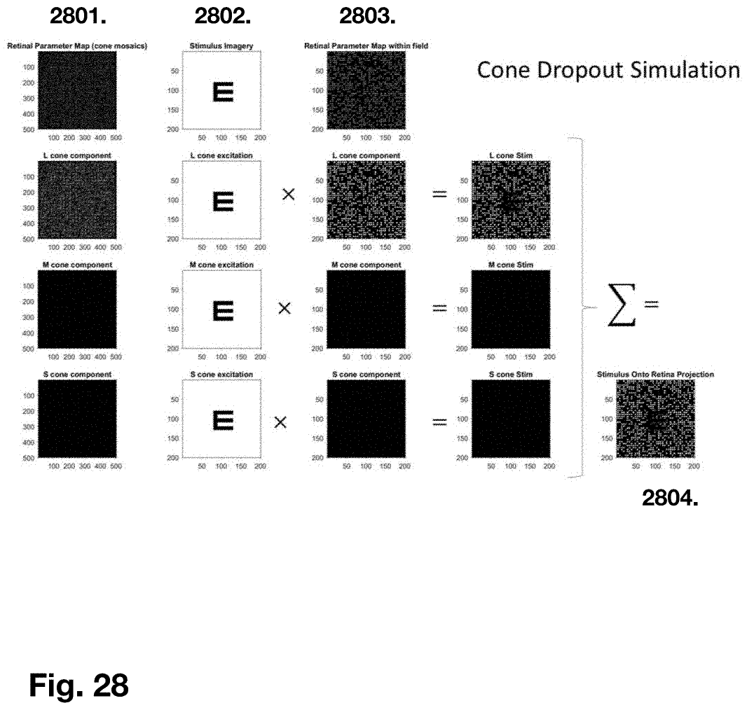

4. Referring to FIG. 28, certain exemplary embodiments of the present invention are directed to test visual acuity with cone dropout. The Stimulus Imagery 2802 would be a letter ‘E’ (same in all layers). The Stimulus Frame would be displayed with gain of 0. The Current Parameter Map 2801 would be a set of cone locations with a random subset removed (read from the Master Retinal Parameter Map). As the eye moves, ICANDI reads the eye position and determines what part of the Current Retinal Parameter Map to read out. The product of the Stimulus Imagery 2802 and the Current Retinal Projection Map 2803 is the Stimulus Onto Retina Projection 2804, which gets played out onto the retina each frame. In effect the E is fixed in world-coordinates and the ‘dropped cones’ travel with the eye motion.

5. Certain exemplary embodiments of the present invention are directed to generate a retinal stimulation signal corresponding to normal trichromatic color vision on the retina of a color-blind person. Without loss of generality, assume in this exemplary embodiment that the user is color-blind in the sense of having only S and L cone cells and lacking M cone cells. This is an exemplary embodiment of Coretsumo. The Stimulus Imagery would be a color image comprised of three layers, the L-component, the M-component and the S-component. The Stimulus Frame would be displayed with a gain of 0. The Current Parameter Map would be a three-channel image containing a stimulation pattern for virtual L1-cones, virtual M-cones and real S-cones (derived from the Master Retinal Parameter Map). The virtual L1-cone map corresponds to a subset of the real L-cone cells, that may be approximately half the real L cells, and approximately evenly distributed relative to the real L cells. The virtual M cone cell map corresponds to the disjunction between the real L cells and the virtual L1-cells. That is, the union of the virtual L1 and M cone cell maps is equal to the real L cone cell map. As the eye moves, ICANDI reads the eye position and determines what part of the Current Retinal Parameter Map to read out. The product of the Stimulus Imagery and the Current Retinal Parameter Map generate three channels which are summed to generate the Stimulus Onto Retina Projection, which gets played out onto the retina. In this exemplary embodiment, the S and L1 cone cells on the retina receive stimuli that may be indistinguishable from the values that the color-blind individual would receive when seeing the color image normally. However, the virtual M cone cells receive the light that a normal-color-vision person would have received on her M cone cells, delivered to the relevant subset of the color-blind individual's L cone cells. In this exemplary embodiment of the present invention the color-blind individual may be able to functionally achieve trichromatic color vision, and may perceive trichromatic colors. One skilled in the art will appreciate how this exemplary embodiment may be modified to treat a color-blind person with missing S or M cone cells, or a person with anomalous color vision, such as M and L cone cells that have spectral response functions that are closer together than in normal color vision. These additional embodiments and more are intended within the general scope of the present invention.

6. Certain exemplary embodiments of the present invention are directed to generate a retinal stimulation signal corresponding to tetrachromatic color vision on the retina of a normal, trichromatic color vision person. Without loss of generality, assume in this exemplary embodiment that the user has normal trichromatic color vision. This is yet another exemplary embodiment of Coretsumo. The Stimulus Imagery would be a color image comprised of four layers. As an illustrative example, consider the four layers to correspond to: the L-component, the M-component, the S-component, and an X-component corresponding to integral projection of incident light's wavelength spectrum on a fourth, virtual photoresponse function, e.g. the L-component shifted to higher wavelengths by 100 nm. The Stimulus Frame would be displayed with a gain of 0. The Current Parameter Map would be a four-channel image containing a stimulation pattern for four real or virtual photoreceptor cells that geometrically coincide with physical photoreceptor cells on the retina. For example, assume that these four virtual receptor types are S1 corresponding to real S cone cell locations; M1 corresponding to real M cone cell locations; L1 corresponding to a subset of the real L-cone cells, that may be approximately half the real L cells, and approximately evenly distributed relative to the real L cells; and X1 corresponding to a subset of cells such that the union of X1 and L1 is equal to the full L map of all real L cone cell locations. Note that S1, M1, L1 and X1 are derived from and added to the Master Retinal Parameter Map. As the eye moves, ICANDI reads the eye position and determines what part of the Current Retinal Parameter Map to read out. The product of the Stimulus Imagery and the Current Retinal Parameter Map generate four channels which are summed to generate the Stimulus Onto Retina Projection, which gets played out onto the retina. In this exemplary embodiment, the S and M cone cells on the retina receive stimuli that may be indistinguishable from the values they would have received when viewing the color image normally. Further, the virtual L1 cone cells receive stimuli corresponding to the values they would have received when viewing the color image normally. However, the virtual X1 cone cells receive a stimuli corresponding to a virtual photoreceptor mosaic with virtual photoresponse function as described above. In this exemplary embodiment of the present invention the user may be able to functionally achieve tetrachromatic color vision, corresponding to S, M, L and X photoreceptor types, and may perceive tetrachromatic colors. One skilled in the art will appreciate how this exemplary embodiment may be modified to inject the X photoresponse image into subsets of the S or M cone cells instead of L, or indeed subsets of cells containing subsets of S, M and L cone cells. These additional embodiments and more are intended within the general scope of the present invention.

7. Following the discussion of the previous implementation details of various exemplary embodiments, numbered 6., one skilled in the art will appreciate how these embodiments may also be modified to define an arbitrary number, N, of different virtual photoreceptor types, with spectral response functions given by X1, X2, . . . , XN, and spatial locations given by subsets of real photoreceptor cells on the retina given by spatial functions XS1, XS2, . . . XSN. And that the eye may be tracked in order to determine the projection of an N dimensional “color” image corresponding to projections of world stimuli onto the photoresponse functions for X1, X2, . . . XN receptor types. And that the movement of the retina may betracked such that this N dimensional image may be projected onto the current location of the retina, against the parameter maps for the relative locations XS1, XS2, . . . XSN of the N virtual photoreceptor types, to create the Stimulus Onto Retina Projection, which gets physically delivered onto the retina. In this exemplary embodiment, the brain of the subject may receive N channels of color-related imagery, and may perceive spectral information of the scene or colors in higher dimensions than regular color vision.

One skilled in the art will recognize that the various exemplary embodiments previously described contain a plurality of sub-component variations and alternatives, all of which are intended to lie within the scope of the present invention, and that these sub-component variations may be permuted and combined in various ways in creating further combinatorial embodiments of the present invention.

As described in detail above, certain exemplary embodiments of the present invention are directed to increasing the dimensionality of color perception for the user. In a specific exemplary embodiment, infrared or thermal imagery is added to the user's perception through the present invention in order to increase the color dimension by one. In this specific exemplary embodiment, this is accomplished by choosing a subset of the cone cells in the retinal map to be labeled as type-IR; receiving infrared imagery, such as from an infrared or thermal camera; mapping the infrared imagery to the retinal maps; computing target values for each cone cell of type-IR according to the value of the infrared image at the corresponding mapped location; and delivering a corresponding light dose to the corresponding cell on the retina as described above. In another exemplary embodiment, the situation is the same as in the previous sentence, except that ultraviolet imagery is received instead of infrared. In yet another exemplary embodiment, an increase by two of the color dimension is accomplished by receiving both infrared and ultraviolet imagery is received, and directed according to the principles described in this paragraph, towards different subsets of cones marked type-IR and type-UV respectively. One skilled in the art may recognize that, according to FIGS. 6, 11 and 16, any number of images, say M, may be utilized in place of infrared and/or ultraviolet, and directed to chosen subsets of cones of type G1, G2, . . . , GM, in order to increase the dimensionality of color vision by M.

In various embodiments of the present invention, imaging and stimulation of the retina are accomplished with an illumination system that can provide both a quality of light suitable for 1-photon imaging for imaging the retina, as well as a quality of light suitable for 2-photon imaging for stimulating the retina. In an exemplary embodiment of the present invention, the illumination system is composed of a continuous wave laser at a certain wavelength for imaging, and a pulsed wave laser (with pulses on the order of, for example, femtoseconds or picoseconds) at the same wavelength, producing a 2-photon effect at an effective wavelength of half that certain wavelength, for stimulation. In a specific exemplary embodiment, the wavelength is 940 nm, which is not visible or weakly visible by a person, and the 2-photon effective wavelength is 470 nm, which can stimulate all three of the S, M and L cone types on the retina. In certain embodiments of the present invention, this type of illumination system, or one that is substantially similar in function, may be utilized in order to limit or eliminate the transverse chromatic aberration offset between the imaging and stimulation lighting on the retina, which may result if the imaging and stimulation light sources are of significantly different wavelength.

In various embodiments of the present invention, general information (e.g. textual, symbolic or sensory) is provided to the retina in a plurality of channels. In some embodiments, the information may be provided in one of the channels of Coretsumo. In other embodiments, it is contained in a plurality of the N channels of information in module 603. In one specific exemplary embodiment, the information is the text of a document, possibly scrolled across the foveal region of the user in an animation, in order for the person to read and become aware of the document's information. In another exemplary embodiment, the information contains the absolute values or changes in a set of chosen stocks. In another exemplary embodiment, the information contains a digital encoding of the on/off states of light switches in a building, encoded as a digital spatial pattern over a set of cone cells in a specific channel of Coretsumo. In yet another exemplary embodiment, the encoded information is the sound contained in an audio file. These exemplary embodiments are intended to illustrate, but not limit, the breadth of general information (e.g. textual, symbolic or sensory) that may be encoded into spatial patterns and delivered to the retina through specific channels of Coretsumo or through a plurality of the N channels of information in module 603.

Note that the present invention is amenable to various modifications and alternative forms, and the drawings and detailed description above illustrate specific versions of such modifications and alternative forms by way of example. It should be understood, however, that the intention is not to limit the invention to the specific embodiments depicted. On the contrary, the intention is to cover all modifications, equivalents, and alternative forms falling within the spirit and scope of the present invention.

### REFERENCES CITED

All references, including patent references, cited herein are hereby incorporated by reference.

### REFERENCES CITED—US PATENTS

U.S. Pat. No. 6,890,076 Roorda
U.S. Pat. No. 7,118,216 Roorda

### REFERENCES CITED—OTHER PUBLICATIONS

Arathorn, D. W., Stevenson, S. B., Yang, Q., Tiruveedhula, P., & Roorda, A. (2013). How the unstable eye sees a stable and moving world. Journal of Vision, 13(10).
Arathorn, D. W., Yang, Q., Vogel, C. R., Zhang, Y., Tiruveedhula, P., & Roorda, A. (2007). Retinally Stabilized Cone-Targeted Stimulus Delivery Optics Express, 15, 13731-13744.
Bedggood, P., & Metha, A. (2017). De-warping of images and improved eyetracking for the scanning laser ophthalmoscope. PLoS One, 12(4), e0174617. doi: 10.1371/journal.pone.0174617
Braaf, B., Vermeer, K. A., Sicam, V. A., van Zeeburg, E., van Meurs, J. C., & deBoer, J. F. (2011). Phase-stabilized optical frequency domain imaging at 1-microm for the measurement of blood flow in the human choroid. Optics Express, 19(21), 20886-20903.
Choi, W., Mohler, K. J., Potsaid, B., Lu, C. D., Liu, J. J., Jayaraman, V., . . . Fujimoto, J. G. (2013). Choriocapillaris and choroidal microvasculature imaging with ultrahigh speed OCT angiography. PLoS One, 8(12), e81499. doi: 10.1371/journal.pone.0081499
Cunefare, D., Fang, L., Cooper, R. F., Dubra, A., Carroll, J., & Farsiu, S. (2017). Open source software for automatic detection of cone photoreceptors in adaptive optics ophthalmoscopy using convolutional neural networks. Sci Rep, 7(1), 6620. doi: 10.1038/s41598-017-07103-0
Dubra, A., Sulai, Y., Norris, J. L., Cooper, R. F., Dubis, A. M., Williams, D. R., & Carroll, J. (2011). Noninvasive imaging of the human rod photoreceptor mosaic using a confocal adaptive optics scanning ophthalmoscope. Biomedical Optics Express, 2(7), 1864-1876.
Harmening, W. M., Tiruveedhula, P., Roorda, A., & Sincich, L. C. (2012). Measurement and correction of transverse chromatic offsets for multi-wavelength retinal microscopy in the living eye. Biomedical Optics Express, 3(9), 2066-2077.
Fairchild, M. “Color Appearance Models, 3rd Edition”, Wiley 2013.
Harmening, W. M., Tuten, W. S., Roorda, A., & Sincich, L. C. (2014). Mapping the perceptual grain of the human retina. Journal of Neuroscience, 34(16), 5667-5677.
Li, K. Y., & Roorda, A. (2007). Automated identification of cone photoreceptors in adaptive optics retinal images. Journal of the Optical Society of America A, 24(5), 1358-1363.
Liang, J., Williams, D. R., & Miller, D. (1997). Supernormal vision and high-resolution retinal imaging through adaptive optics. Journal of the Optical Society of America A, 14(11), 2884-2892.
Mainster, M. A., Timberlake, G. T., Webb, R. H., & Hughes, G. W. (1982). Scanning laser ophthalmoscopy. Clinical applications. Ophthalmology, 89(7), 852-857.
Merino, D., Duncan, J. L., Tiruveedhula, P., & Roorda, A. (2011). Observation of cone and rod photoreceptors in normal subjects and patients using a new generation adaptive optics scanning laser ophthalmoscope. Biomedical Optics Express, 2(8), 2189-2201.
Mulligan, J. B. (1997). Recovery of motion parameters from distortions in scanned images. Proceedings of the NASA Image Registration Workshop (IRW97) NASA Goddard Space Flight Center, MD.
Poonja, S., Patel, S., Henry, L., & Roorda, A. (2005). Dynamic visual stimulus presentation in an adaptive optics scanning laser ophthalmoscope. Journal of Refractive Surgery, 21(5), S575-S580.
Ratnam, K., Domdei, N., Harmening, W. M., & Roorda, A. (2017). Benefits of retinal image motion at the limits of spatial vision. Journal of Vision, 17(1), 30. doi: 10.1167/17.1.30
Roorda, A., Romero-Bora, F., Donnelly, W. J., Queener, H., Hebert, T. J., & Campbell, M. C. W. (2002). Adaptive optics scanning laser ophthalmoscopy. Optics Express, 10(9), 405-412.
Roorda, A., & Williams, D. R. (1999). The arrangement of the three cone classes in the living human eye. Nature, 397, 520-522.
Sabesan, R., Hofer, H., & Roorda, A. (2015). Characterizing the Human Cone Photoreceptor Mosaic via Dynamic Photopigment Densitometry. PLoS One, 10(12), e0144891. doi: 10.1371/journal.pone.0144891
Sheehy, C. K., Arathorn, D. W., Yang, Q., Tiruveedhula, P., & Roorda, A. (2012). High-speed, Image-based Eye Tracking With A Scanning Laser Ophthalmoscope. ARVO Meeting Abstracts, 53(6), 3086.
Stetter, M., Sendtner, R. A., & Timberlake, G. T. (1996). A novel method for measuring saccade profiles using the scanning laser ophthalmoscope. Vision Research, 36(13), 1987-1994.
Stevenson, S. B., & Roorda, A. (2005). Correcting for miniature eye movements in high resolution scanning laser ophthalmoscopy. In F.
Manns, P. Soderberg, & A. Ho (Eds.), Ophthalmic Technologies XI (pp. 145-151). Bellingham, Wash.: SPIE. (Reprinted from: NOT IN FILE).
Sulai, Y. N., Scoles, D., Harvey, Z., & Dubra, A. (2014). Visualization of retinal vascular structure and perfusion with a nonconfocal adaptive optics scanning light ophthalmoscope. Journal of the Optical Society of America A, 31(3), 569-579.
Tam, J., Martin, J. A., & Roorda, A. (2010). Noninvasive visualization and analysis of parafoveal capillaries in humans. Investigative Ophthalmology and Visual Science, 51(3), 1691-1698.
Timberlake, G. T., Mainster, M. A., Webb, R. H., Hughes, G. W.,& Trempe, C. L. (1982). Retinal localization of scotomata by scanning laser ophthalmoscopy. Investigative Ophthalmology and Visual Science, 22(1), 91-97.
Tuten, W. S., Tiruveedhula, P., & Roorda, A. (2012). Adaptive optics scanning laser ophthalmoscope-based microperimetry. Optometry and Vision Science, 89(5), 563-574.
Vogel, C. R., Arathorn, D. W., Roorda, A., & Parker, A. (2006). Retinal motion estimation and image dewarping in adaptive optics scanning laser ophthalmoscopy. Optics Express, 14(2), 487-497.
Webb, R. H., & Hughes, G. W. (1981). Scanning laser ophthalmoscope. IEEE Transactions on Biomedical Engineering, 28, 488-492.
Webb, R. H., Hughes, G. W., & Pomerantzeff, O. (1980). Flying spot TV ophthalmoscope. Applied Optics, 19, 2991-2997.
Yang, Q., Arathorn, D. W., Tiruveedhula, P., Vogel, C. R., & Roorda, A. (2010). Design of an integrated hardware interface for AOSLO image capture and cone-targeted stimulus delivery. Optics Express, 18(17), 17841-17858.

According to an embodiment, a method of stimulating a retina of an eye is provided. The method includes mapping the retina to determine a map of the retina, defining a retinal parameter map by assigning one or more parameters to positions on the map of the retina, receiving an image signal, and calculating, based on the image signal and the retinal parameter map, stimulus values to be applied to each of a plurality of photoreceptors of the retina.

According to another embodiment, a method of stimulating a retina of an eye is provided. The method includes mapping the retina to determine a map of the retina, defining a retinal parameter map by assigning one or more parameters to positions on the map of the retina, receiving an image signal, calculating, based on the image signal and the retinal parameter map, stimulus values to be applied to each of a plurality of photoreceptors of the retina, and physically delivering stimulus to the plurality of photoreceptors based on the calculated stimulus values.

According to yet another embodiment, a method of stimulating a retina of an eye is provided. The method includes mapping the retina to determine a map of the retina, defining a retinal parameter map by assigning one or more parameters to positions on the map of the retina, tracking a relative movement of the eye to determine eye tracking information, receiving an image signal, computing, based on the image signal and the eye tracking information, a transformation of the image signal onto the map of the retina, calculating, based on the transformation and the retinal parameter map, stimulus values to be applied to each of a plurality of photoreceptors of the retina, and physically delivering stimulus to the plurality of photoreceptors based on the calculated stimulus values and the eye tracking information. The stimulus delivered to the plurality of photoreceptors represents a color outside of the natural human color gamut, or one or more color channels missing in a vision system of a color blind person, or an image channel not normally viewable by the eye.

In further embodiments, a method may include selecting a subset of the plurality of photoreceptors as virtual photoreceptors, the virtual receptors corresponding to locations on the map of the retina, wherein the calculating stimulus values includes mapping the image signal to locations on the map of the retina, and computing a target stimulus value for each of the virtual photoreceptors based on a value of the image signal at the corresponding mapped location.

According to a further embodiment, a system for stimulating a retina of an eye is provided. The system includes a retina mapper configured to determine a map of the retina, a retinal map parameter assigner configured to define a retinal parameter map by assigning one or more parameters to positions on the map of the retina, an image data creator configured to receive and/or create an image signal, a retinal stimulus calculator configured to calculate, based on the image signal and the retinal parameter map, stimulus values to be applied to each of a plurality of photoreceptors of the retina, and a stimulus delivery device configured to physically deliver stimulus to the plurality of photoreceptors based on the calculated stimulus values.

In certain embodiments, the retinal map parameter assigner, the image data creator, and the retinal stimulus calculator are implemented together in one or more processing devices. In certain embodiments, the retinal map parameter assigner, the image data creator, and the retinal stimulus calculator are each separately implemented in one or more processing devices.

In certain embodiments, a non-transitory computer readable medium is provided that includes code, which when executed by one or more processors, causes the one or more processors to interface with various devices and implement the various methods, or aspects of the various methods, as described herein. Such a computer readable medium may be embodied as a physical storage device or medium such as a CD, DVD, thumb drive, ROM memory, RAM memory or the like.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Exemplary embodiments are described herein. Variations of those exemplary embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of stimulating a retina of an eye, the method comprising:
mapping the retina to determine a map of the retina;
defining a retinal parameter map by assigning one or more parameters to positions on the map of the retina;
receiving an image signal;
calculating, based on the image signal and the retinal parameter map, stimulus values to be applied to each of a plurality of photoreceptors of the retina; and
physically delivering stimulus to the plurality of photoreceptors based on the calculated stimulus values.

2. The method of claim 1, wherein mapping the retina includes scanning the retina with an adaptive optics scanning laser ophthalmoscope (AOSLO) to image the retina.

3. The method of claim 1, wherein the one or more parameters include one or more of a biological type of photosensitive cells of the retina, a virtual photoreceptor type of the photosensitive cells of the retina, and a virtual spectral responsivity of the photosensitive cells of the retina.

4. The method of claim 1, wherein the receiving an image signal includes receiving and/or creating one of an RGB image or video, a hyper-spectral image or video, a grayscale image or video or a full color image or video.

5. The method of claim 1, further including tracking a relative movement of the eye to determine eye tracking information.

6. The method of claim 5, wherein calculating stimulus values includes computing, based on the image signal and the eye tracking information, a transformation of the image signal onto the map of the retina.

7. The method of claim 6, wherein computing the transformation includes mapping display coordinates onto the map of the retina.

8. The method of claim 1, wherein the calculating stimulus values includes determining or calculating a stimulus value for each position on the map of the retina based on a biological type of a photosensitive cell at the position or based on a photoresponse function of the photosensitive cell at the position.

9. The method of claim 1, wherein physically delivering stimulus to the retina includes scanning the retina with an adaptive optics scanning laser ophthalmoscope (AOSLO) to stimulate the retina.

10. The method of claim 1, further comprising selecting a subset of the plurality of photoreceptors as virtual photoreceptors, the virtual receptors corresponding to locations on the map of the retina, wherein the calculating stimulus values includes:
mapping the image signal to locations on the map of the retina; and
computing a target stimulus value for each of the virtual photoreceptors based on a value of the image signal at the corresponding mapped location.

11. The method of claim 10, wherein the plurality of photoreceptors includes S-type, M-type and L-type cone cells of the eye, and wherein the virtual photoreceptors represent at least one type of the S-type, M-type and/or L-type cone cells.

12. The method of claim 10, wherein the target stimulus values delivered to the virtual receptors represent one of;
a color outside of the natural human color gamut;
one or more color channels missing in a vision system of a color blind person;
and
an image channel not normally viewable by the eye.

13. (canceled)

14. (canceled)

15. The method of claim 10, wherein the image channel includes one of an infrared image channel or an ultraviolet image channel.

16. A system for stimulating a retina of an eye, the system comprising: a retina mapper configured to determine a map of the retina;
a retinal map parameter assigner configured to define a retinal parameter map by assigning one or more parameters to positions on the map of the retina;
an image data creator configured to receive and/or create an image signal;
a retinal stimulus calculator configured to calculate, based on the image signal and the retinal parameter map, stimulus values to be applied to each of a plurality of photoreceptors of the retina; and
a stimulus delivery device configured to physically deliver stimulus to the plurality of photoreceptors based on the calculated stimulus values.

17. The system of claim 16, wherein the retinal map parameter assigner, the image data creator, and the retinal stimulus calculator are implemented together in one or more processing devices.

18. The system of claim 16, wherein the retinal map parameter assigner, the image data creator, and the retinal stimulus calculator are each separately implemented in one or more processing devices.

19. The system of claim 16, wherein the retina mapper includes an adaptive optics scanning laser ophthalmoscope (AOSLO) configured to image the retina.

20. The system of claim 16, wherein the stimulus delivery device includes an adaptive optics scanning laser ophthalmoscope (AOSLO) configured to stimulate the retina.

21.-33. (canceled)

34. A method of stimulating a retina of an eye, the method comprising: mapping the retina to determine a map of the retina;
defining a retinal parameter map by assigning one or more parameters to positions on the map of the retina;
tracking a relative movement of the eye to determine eye tracking information; receiving an image signal;
computing, based on the image signal and the eye tracking information, a transformation of the image signal onto the map of the retina;
calculating, based on the transformation and the retinal parameter map, stimulus values to be applied to each of a plurality of photoreceptors of the retina; and
physically delivering stimulus to the plurality of photoreceptors based on the calculated stimulus values and the eye tracking information;
wherein the stimulus delivered to the plurality of photoreceptors represents a color outside of the natural human color gamut, or one or more color channels missing in a vision system of a color blind person, or an image channel not normally viewable by the eye.

35. The method of claim 34, wherein the steps of mapping and physically delivering stimulus are each performed using a device capable of imaging and/or stimulating the retina at a per-photoreceptor accuracy.
